(19) United States
(12) Patent Application Publication
(10) Pub. No.: US 2020/0398004 A1
(43) Pub. Date: Dec. 24, 2020

(54) Inhaler and Methods of Use Thereof

(71) Applicant: Microdose Therapeutx, Inc., Ewing, NJ (US)

(72) Inventors: Robert Leonard, Jamison, PA (US); John P. Majewski, Riverton, NJ (US); Douglas Weitzel, Hamilton, NJ (US)

(73) Assignee: Microdose Therapeutx, Inc., Ewing, NJ (US)

(21) Appl. No.: 16/975,008

(22) PCT Filed: Feb. 22, 2019
(86) PCT No.: PCT/US2019/019138

§ 371 Date: Aug. 21, 2020

Related U.S. Application Data
(60) Provisional application No. 62/634,555, filed on Feb. 23, 2018.

(51) Int. Cl. A61M 15/00

(52) U.S. Cl. CPC A61M 15/0065; A61M 15/0086; A61M 2202/064; A61M 2205/583; A61M 2205/3334; A61M 15/0091

(57) ABSTRACT

A single-dose cartridge inhaler comprises a dosing chamber configured to contain dry powder medicament, a transducer and a controller electrically coupled to the transducer. The medicament delivery device is capable of delivering a therapeutically effective dose of dry powder medicament in response to between 2-20 tidal inhalations, the dose preferably having a mass median aerodynamic diameter (MMAD) of about 6 microns or less and a fine particle fraction of at least 30%.

### CROSS-REFERENCE TO RELATED APPLICATIONS

This application claims the benefit of U.S. Provisional Patent Application No. 62/634,555 filed Feb. 23, 2018 entitled “Inhaler and Methods of Use Thereof”, which is incorporated by reference herein in its entirety.

### FIELD OF THE INVENTION

The present invention relates to a device for administering medicament. In particular, the invention relates to a device for use in administering medicament in powder form.

### BACKGROUND OF THE INVENTION

Certain diseases and disorders of the respiratory tract are known to respond to treatment by the direct application of therapeutic agents. As these agents are most readily available in dry powder form, their application is most conveniently accomplished by inhaling the powdered material through the nose or mouth. This powdered form results in better utilization of the medication, as the drug is deposited at the site where its action is needed; hence, very small doses of the drug are often as efficacious as larger doses administered orally or by injection, with a consequent marked reduction in the incidence of undesired side effects and medication cost. Alternatively, a drug in powder form may be used for the treatment of diseases and disorders other than those of the respiratory system. When the drug is deposited on the large surface areas of the lungs, it may be rapidly absorbed into the blood stream; hence, this method of application may take the place of administration by injection, tablet, or other conventional means.

Dry powder inhalers (DPI's) of the prior art have means for introducing a drug formulation into an air stream. Several inhalation devices useful for dispensing a powder form of medication are known in the prior art. For example, U.S. Pat. Nos. 2,517,482; 3,507,277; 3,518,992; 3,635,219; 3,795,244; 3,807,400; 3,831,606; 3,948,264; and 5,458,135 describe inhalation devices, many of which have means for piercing or removing the top of a capsule containing a powdered medication. Several of these patents disclose propeller means, which aid in dispensing the powder out of the capsule. Other DPI's utilize a vibratory element, such as those described in U.S. Pat. Nos. 5,694,920; 6,026,809; 6,142,146; 6,152,130; 7,080,644 and 7,318,434.

The prior art devices possess several disadvantages. For example, they often require that the user exert considerable effort in inhalation to effect withdrawal of the powder into the inhaled air stream. Thus, their performance often heavily depends on the flow rate generated by the user—a low flow rate may not result in the powder being sufficiently deaggregated, which can cause uncontrolled amounts or clumps of powder being inhaled into the user's mouth, rather than a constant inhalation of controlled amounts of finely dispersed drug. This adversely affects the dose delivered to the patient and can lead to inconsistency in the bioavailability of the drug from dose-to-dose due to lack of consistency in the deaggregation process. Consequently, patients that cannot produce sufficiently high flow rates, such as pediatric, elderly, and patients with severely compromised lung function (e.g., COPD), may receive reduced and/or variable doses at the intended site of delivery. Moreover, suction of powder through the pierced holes of a capsule by inhalation often does not withdraw all or even most of the powder out of a capsule, thus causing a waste of the medication. The large energy requirements for driving electromechanical based inhalers typically increase the size of the devices, making them unsuitable for portable use.

Nebulizers provide an alternative mechanism for delivering medication to the respiratory system in a manner that may not require forceful inspiration. However, current nebulization systems are limited by relatively slow drug delivery; for example, some systems require a session of at least 10-20 minutes. This is especially undesirable for patients that regularly use a nebulizer several times per day. Also, nebulizers typically lack portability, are cumbersome to set up, and require a significant amount of cleaning and maintenance, among other drawbacks.

Efficient delivery of inhaled medication is desirable for the success of pulmonary-delivered therapies. One of the most desirable factors in pulmonary delivery from a DPI is a high-quality aerosol, in terms of the aerosol's aerodynamic particle size, and its potential to consistently achieve the desired lung deposition in vivo. The optimal delivery of inhaled medications is hindered in current devices by the need for patients to inhale forcefully while coordinating inspiration with the device, as well as by the physical limitations of the patient. Devices that provide means for deaggregating the powder have not been shown to provide consistent dose delivery or particle size distribution. These problems highlight the significant unmet need for simpler, portable, easier-to-use devices that do not require coordination with forceful inspiration, provide a short duration of administration, and deagglomerate the drug formulation in a manner that ensures a consistent particle size distribution of the delivered dose throughout the life of the device.

### BRIEF SUMMARY OF THE INVENTION

In one embodiment, a medicament delivery device comprises a dosing chamber comprising an interior that is configured to contain dry powder medicament, a transducer confronting the dosing chamber, wherein the dosing chamber and the transducer are acoustically resonant such that the dosing chamber is configured to resonate in response to an activation of the transducer, and a controller electrically coupled to the transducer and configured to send an electrical signal that activates the transducer when the medicament delivery device senses a subject's dosing breath. The medicament delivery device preferably has a flow resistance in a range from about 0.040 cmH₂O^{0.5}/LPM to about 0.1 cmH₂O^{0.5}/LPM at 30 liters per minute (LPM). The device is preferably capable of delivering a therapeutically effective dose of dry powder medicament in response to between 2-20 tidal inhalations. The device preferably comprises a base and a removable single-dose cartridge, wherein the dosing chamber is contained within the removable single-dose cartridge, and the dose of dry powder medicament is contained inside the dosing chamber prior to delivery of the dose.

In one embodiment, the controller is configured to activate the transducer for a total on-time of 5 seconds or less over the 2-20 tidal inhalations.

In one embodiment, the controller is configured to activate the transducer for between about 50 ms and about 1000 ms during each dosing breath.

In one embodiment, the medicament delivery device is capable of delivering the dose of dry powder medicament at flow rates at least within a range of about 15 LPM to about 30 LPM.

In one embodiment, the medicament delivery device is capable of delivering the dose of dry powder medicament at flow rates at least within a range of about 15 LPM to about 90 LPM.

In one embodiment, the medicament delivery device is configured to administer at least 10% of the dry powder medicament dose in response to a first dosing breath.

In one embodiment, the dose has a mass median aerodynamic diameter (MMAD) of about 6 microns or less and a fine particle fraction of at least 30%.

In one embodiment, the amount of the dose of dry powder medicament is between about 1 mg and about 100 mg.

In one embodiment, the device comprises one or more lights configured to illuminate when a dose has been administered

According to another embodiment, a method of treating a respiratory disease or condition (e.g., COPD, asthma, CF, IPF, etc.), or one or more symptoms thereof (e.g., a method of increasing a subject's FEV₁) comprises inhaling a therapeutically effective dose of dry powder medicament through a medicament delivery device using between 2-20 tidal inhalations over the course of an inhalation cycle, the inhalation cycle comprising dosing breaths, wherein the medicament delivery device comprises a vibratory element that is activated upon each dosing breath and causes dry powder medicament to be aerosolized and expelled from one or more openings in a dosing chamber into an air flow conduit, wherein the medicament delivery device comprises a base and a removable single-dose cartridge, the dosing chamber is contained within the removable single-dose cartridge, and the dose of dry powder medicament is contained inside the dosing chamber prior to the delivery of the dose. The medicament delivery device preferably has a flow resistance in a range from about 0.040 cmH₂O^{0.5}/LPM to about 0.1 cmH₂O^{0.5}/LPM at 30 liters per minute (LPM) and is capable of delivering the dose of dry powder medicament in response to tidal inhalation (e.g., in response to flow rates at least within a range of about 15 LPM to about 30 LPM). Preferably, the dose of dry powder medicament delivered by the medicament delivery device has a mass median aerodynamic diameter (MMAD) of about 6 microns or less and a fine particle fraction of at least 30%. Preferably, the dose of medicament is delivered within 60 seconds or less, or within 45 seconds or less, or within 30 seconds or less. Preferably, the medicament delivery device is configured to administer at least about 10%, or at least about 15%, or at least about 20%, or at least about 25%, or at least about 30%, or at least about 40%, or at least about 50%, or at least about 60% of the dry powder medicament dose in response to a first dosing breath in an inhalation cycle.

In one embodiment, the method further comprises exhaling away from the medicament delivery device after each tidal inhalation.

In one embodiment, the dose of dry powder medicament administered by the medicament delivery device has a mass median aerodynamic diameter (MMAD) of about 6 microns or less and a fine particle fraction of at least 30%.

In one embodiment, the medicament delivery device administers at least 10% of the dry powder medicament dose in response to the first dosing breath in the inhalation cycle.

In one embodiment, the transducer has an on-time of about 5 seconds or less over the course of the inhalation cycle.

In one embodiment, the medicament delivery device achieves maximum synthetic jetting within about 1000 ms or less from the start of each transducer activation.

In one embodiment, the dose of dry powder medicament is administered within 1 minute or less.

In one embodiment, the respiratory disease or condition is COPD.

In one embodiment, the respiratory disease or condition is COPD and the dry powder medicament comprises a LAMA and a LABA.

In one embodiment, the respiratory disease or condition is COPD and the dry powder medicament comprises glycopyrronium bromide and formoterol fumarate.

In one embodiment, the respiratory disease or condition is asthma.

In one embodiment, the respiratory disease or condition is cystic fibrosis and the dry powder medicament comprises one or more antibiotics.

In one embodiment, the respiratory disease or condition is cystic fibrosis and the dry powder medicament comprises DNase (e.g., dornase alfa).

In one embodiment, the respiratory disease or condition is idiopathic pulmonary fibrosis and the dry powder medicament comprises pirfenidone.

According to another embodiment, a method of treating a respiratory disease or condition (e.g., COPD, asthma, CF, IPF, etc.), or one or more symptoms thereof (e.g., a method of increasing a subject's FEV₁) comprises inhaling a therapeutically effective dose of dry powder medicament through a medicament delivery device using between 2-20 tidal inhalations over the course of an inhalation cycle, the inhalation cycle comprising dosing breaths, wherein the medicament delivery device comprises a vibratory element that is activated upon each dosing breath and causes dry powder medicament to be aerosolized and expelled from one or more openings in a dosing chamber into an air flow conduit, wherein the medicament delivery device comprises a base and a removable single-dose cartridge, the dosing chamber is contained within the removable single-dose cartridge, and the dose of dry powder medicament is contained inside the dosing chamber prior to delivery of the dose. The medicament delivery device may comprise a high-velocity flow channel, the high-velocity flow channel comprising a constricted section disposed over the one or more openings in the dosing chamber, or adjacent to the one or more openings in the dosing chamber, wherein the dose of medicament is expelled over the course of the inhalation cycle by a combination of active delivery and passive delivery, and wherein the medicament delivery device is capable of delivering the dose of dry powder medicament in response to tidal inhalation (e.g., in response to flow rates at least within a range of about 15 LPM to about 30 LPM). The medicament delivery device preferably has a flow resistance in a range from about 0.040 cmH₂O^{0.5}/LPM to about 0.1 cmH₂O^{0.5}/LPM at 30 liters per minute (LPM). Preferably, the dose of dry powder medicament delivered by the medicament delivery device has a mass median aerodynamic diameter (MMAD) of about 6 microns or less and a fine particle fraction of at least 30%. Preferably, the dose of medicament is delivered within 60 seconds or less, or within 45 seconds or less, or within 30 seconds or less.

### BRIEF DESCRIPTION OF THE SEVERAL VIEWS OF THE DRAWINGS

The foregoing summary, as well as the following detailed description of embodiments of the device and methods of use, will be better understood when read in conjunction with the appended drawings of particular embodiments. It should be understood, however, that the invention is not limited to the precise arrangements and instrumentalities shown. It will also be appreciated that the drawings show merely schematic representation of possible embodiments of a device in accordance with the invention; for example, the shape of the device illustrated is not essential to the present invention, and alternative embodiments of the device could look different from the views shown in the drawings.

In the drawings:

FIG. 1 is a graph showing transducer pulse duration in relation to a breathing cycle;

FIG. 2 illustrates an inhaler in accordance with one embodiment of the present invention;

FIGS. 3A, 3B and 3C are schematic diagrams of embodiments of a medicament delivery device comprising a base and a single-use cartridge.

FIG. 4 is a close-up sectional view of a top portion of the inhaler of FIG. 2 along a plane defined by line 1-1;

FIG. 5 illustrates an embodiment of a dosing chamber having nodes (N) and anti-nodes (A);

FIG. 6 illustrates an embodiment of a dosing chamber with an internal height X;

FIG. 7A illustrates an embodiment of a dosing chamber having a larger internal height compared to the embodiment of the dosing chamber shown in FIG. 7B;

FIG. 8A illustrates a side view of an embodiment of a dosing chamber having an apex;

FIG. 8B illustrates a side view of an embodiment of a dosing chamber without an apex;

FIG. 9 is a side view of an embodiment of a base;

FIG. 10 illustrates an embodiment of a base and single-use cartridge;

FIG. 11 illustrates a sectional view of the embodiment of the single-use cartridge shown in FIG. 10;

FIG. 12 illustrates an embodiment of a single-use cartridge;

FIG. 13 illustrates an embodiment of a dosing chamber;

FIG. 14 is a front elevational view of a membrane in accordance with one embodiment of the present invention;

FIG. 15 is a side elevational view of the membrane of FIG. 14;

FIG. 16 illustrates an embodiment of the air flow conduit;

FIGS. 17A, 17B and 17C illustrate embodiments of the air flow conduit also referred to as high-velocity flow channels;

FIG. 18 is a schematic diagram of an inhaler property observation rig in accordance with one embodiment of the present invention;

FIG. 19 is a schematic diagram of a system for measuring flow resistance in accordance with one embodiment of the present invention;

FIG. 20 illustrates an embodiment of a single-use cartridge comprising a sealing mechanism that has been moved away from the dosing chamber openings; and

FIG. 21 illustrates an embodiment of a transducer comprising a spacer.

### DETAILED DESCRIPTION OF THE INVENTION

The present invention relates to a device for administering medicament as a dry powder for inhalation by a subject. Some embodiments of the device may be classified as a dry powder inhaler (DPI). Some embodiments of the device may also be classified as a dry powder nebulizer (as opposed to a liquid nebulizer), particularly when tidal breathing (e.g., tidal inhalation) is used to deliver dry powder medicament over multiple inhalations. The device may be referred to herein interchangeably as a “medicament delivery device” or an “inhaler,” both of which refer to a device for administering medicament as a dry powder for inhalation by a subject, preferably over multiple inhalations, and most preferably when tidal inhalation is used. “Tidal breathing” preferably refers to inhalation and exhalation during normal breathing at rest, as opposed to forceful breathing. Similarly, “tidal inhalation” refers to normal inhalation at rest, as opposed to inhalation that requires extra effort on the part of the user, such as forceful inhalation at a high inspiratory flow, or slow, deep inhalation. Stated another way, inhalation that requires extra effort may include inhalation that is slower, deeper, faster or stronger than normal inhalation at rest, whereas tidal inhalation refers to normal inhalation at rest which requires no extra effort.

A preferred embodiment of the medicament delivery device described herein may be referred to as a single-dose cartridge inhaler, or an inhaler having a single-use cartridge. In accordance with this embodiment, the device comprises a base and an attachable cartridge, wherein the dosing chamber is contained within the attachable cartridge, and the dose of dry powder medicament is contained inside the dosing chamber prior to delivery of the dose to a subject. Thus, the cartridge preferably contains a single dose of medicament inside the dosing chamber, instead of a blister strip comprising multiple doses. According to certain embodiments, the dosing chamber may contain a dose of dry powder medicament ranging from about 5 mg to about 250 mg, preferably from about 5 mg to about 80 mg.

As used herein, the term therapeutically effective amount may refer to an amount that, when administered to a particular subject, achieves a therapeutic effect by inhibiting, alleviating or curing a disease, disorder or symptom(s) in the subject or by prophylactically inhibiting, preventing or delaying the onset of a disease, disorder or symptom(s). A therapeutically effective amount may be an amount which relieves to some extent one or more symptoms of a disease or disorder in a subject; and/or returns to normal either partially or completely one or more physiological or biochemical parameters associated with or causative of the disease or disorder; and/or reduces the likelihood of the onset of the disease, disorder or symptom(s).

The terms medicament, pharmaceutical, active agent, active pharmaceutical ingredient, API, drug, medication, and active are used herein interchangeably to refer to the pharmaceutically active compound(s) in the drug composition. Other ingredients in a drug composition, such as carriers or excipients, may be substantially or completely pharmaceutically inert. A drug composition (also referred to herein as a composition, formulation, drug formulation, pharmaceutical composition, medicament formulation or API formulation) may comprise the medicament in combination with one or more carriers and/or one or more excipients. Some examples of suitable medicaments in accordance with the present invention include those that treat respiratory diseases or disorders. Non-limiting examples of respiratory diseases and disorders include chronic obstructive pulmonary disease (COPD) (including chronic bronchitis and/or emphysema), asthma, bronchitis, cystic fibrosis, idiopathic pulmonary fibrosis and chest infections such as pneumonia.

The term “pharmaceutically acceptable,” as used herein, means permitted by a regulatory agency, e.g. of a European or U.S. Federal or state government, or listed in the U.S. Pharmacopeia or other generally recognized pharmacopeia for use in animals, and more particularly in humans.

The terms user, subject and patient are used interchangeably herein and may refer to a mammalian individual, preferably a human being.

The terms micrometers, microns and μm may be used interchangeably. The terms micrograms, mcg and μg may be used interchangeably.

As used herein, the terms respiratory diseases and disorders may be used interchangeably with pulmonary diseases and disorders, respectively.

Each compound used herein may be discussed interchangeably with respect to its chemical formula, chemical name, abbreviation, etc. For example, glycopyrronium bromide may be used interchangeably with glycopyrrolate.

Embodiments of the medicament delivery device of the present invention (also referred to herein as an inhaler) are capable of delivering doses of dry powder medicament in consistent amounts with consistent particle size distributions over a wide range of breathing patterns and flow rates. For example, embodiments of the inhaler can deliver consistent doses to patients that use regular breathing patterns (e.g., tidal breathing or tidal inhalation) to trigger the delivery of medication, and forceful inspiration is not needed. According to a preferred embodiment, the medicament delivery device of the present invention delivers substantially uniform doses and particle size distributions over a wide range of flow rates.

According to preferred embodiments, the inhaler detects inhalation and administers medicament in response to the detected inhalation, whereby aerosolized medicament is released into the air flow conduit and becomes entrained into the subject's inhaled air. As described in more detail below, this is preferably achieved through the use of a vibration means (or “vibrating element”) for aerosolizing and releasing material into an air flow conduit, wherein the vibrating element preferably creates mechanical vibrations and acoustic vibrations that aerosolize the medicament via synthetic jetting.

According to an embodiment, a user inhales through the mouthpiece of the device, preferably via tidal inhalation, and the dose is delivered over a plurality of consecutive inhalations. Thus, in one embodiment, the inhaler is configured to activate a transducer more than once to deliver a complete pharmaceutical dose to a user. When the user inhales through the mouthpiece, air is drawn into the device's air inlet, through an air flow conduit in the device, and out of the mouthpiece into the user's lungs. As air is being inhaled through the air flow conduit, dry powder medicament is expelled into the airflow pathway and becomes entrained in the user's inhaled air. Thus, the air flow conduit preferably defines an air path from the air inlet to the outlet (i.e., the opening that is formed by the mouthpiece). Each breath cycle includes an inhalation and an exhalation, i.e., each inhalation is followed by an exhalation, so consecutive inhalations preferably refer to the inhalations in consecutive breath cycles. After each inhalation, the user may either exhale back into the mouthpiece of the inhaler, or exhale outside of the inhaler (e.g., by removing his or her mouth from the mouthpiece and expelling the inhaled air off to the side). Preferably, the user exhales outside the inhaler.

Embodiments of an inhaler comprising a blister strip are described in U.S. patent application Ser. No. 15/729,526, which is incorporated by reference herein, in which pre-metered doses of dry powder medicament may be stored in a blister strip that is contained in a detachable cartridge. An embodiment of a blister advance mechanism is described in US 2016/0296717, which is incorporated by reference herein.

A blister strip and its dose advance mechanism are not required in accordance with embodiments of the present invention. According to a preferred embodiment described in more detail below, a dose of dry powder medicament is initially stored and contained in the dosing chamber, in which case the blister strip and its advance mechanism are not required. This embodiment may be referred to as a single-dose cartridge inhaler or an inhaler having a single-use cartridge. According to an exemplary embodiment of the present invention, the inhaler comprises a reusable base 102 and an attachable single-use cartridge 103, wherein the dosing chamber is disposed in the cartridge 103 and a single dose of dry powder medicament is contained inside the dosing chamber. Thus, the single-dose cartridge inhaler preferably comprises a reusable component (also referred to herein as a base or back portion) that attaches to a replaceable component (also referred to herein as a cartridge or front portion), wherein the replaceable component may comprise a single dose of dry powder medicament contained inside the dosing chamber. Preferably, the reusable cartridge comprises the dosing chamber and air flow conduit (also referred to as a flow channel). A new cartridge may be attached to the base each time a subject needs to take a dose of medicament, and subsequently removed from the base and discarded after the dose is administered.

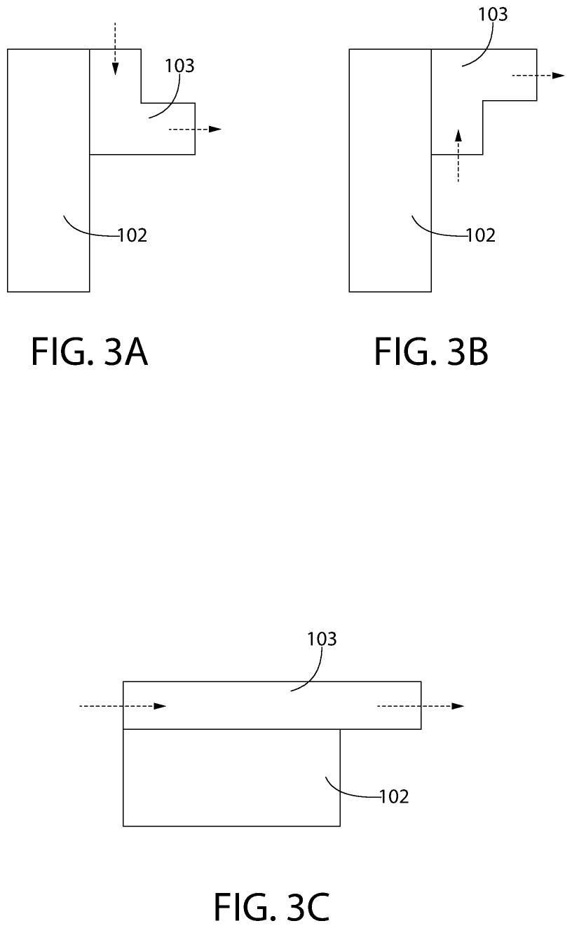

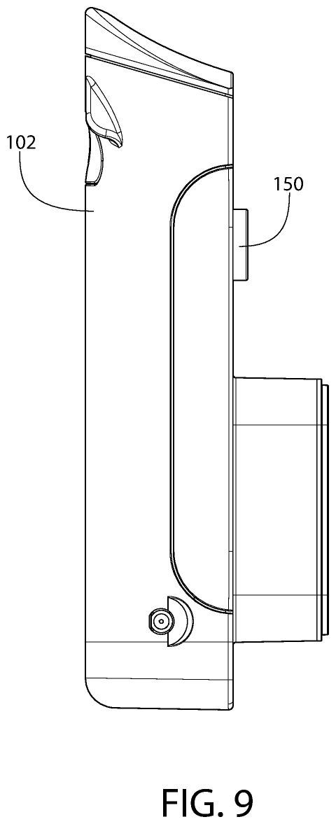

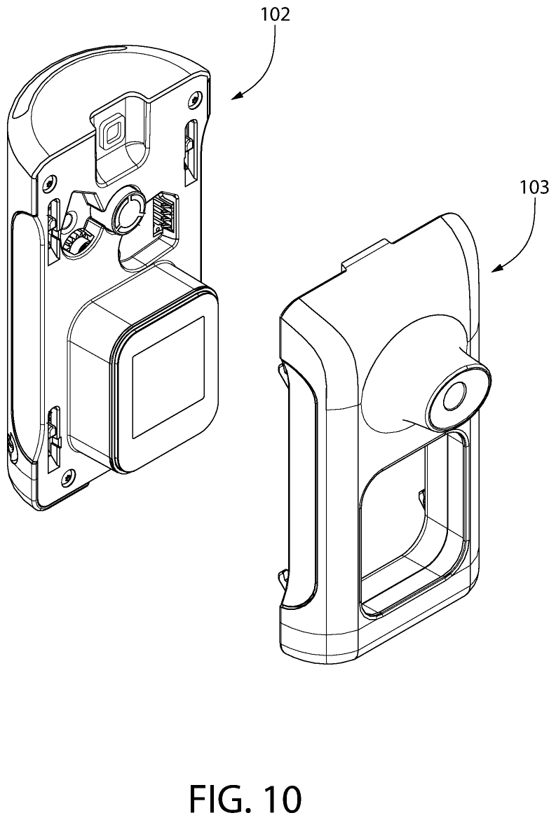

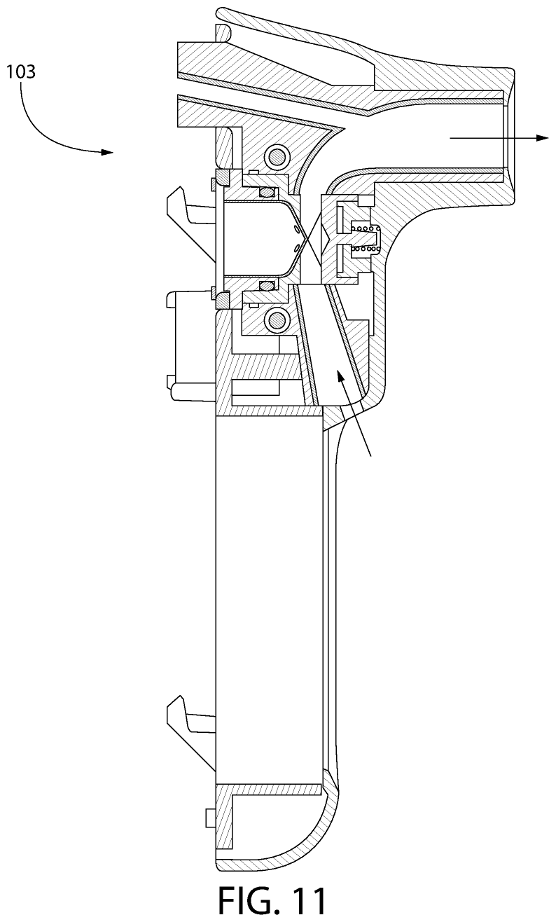

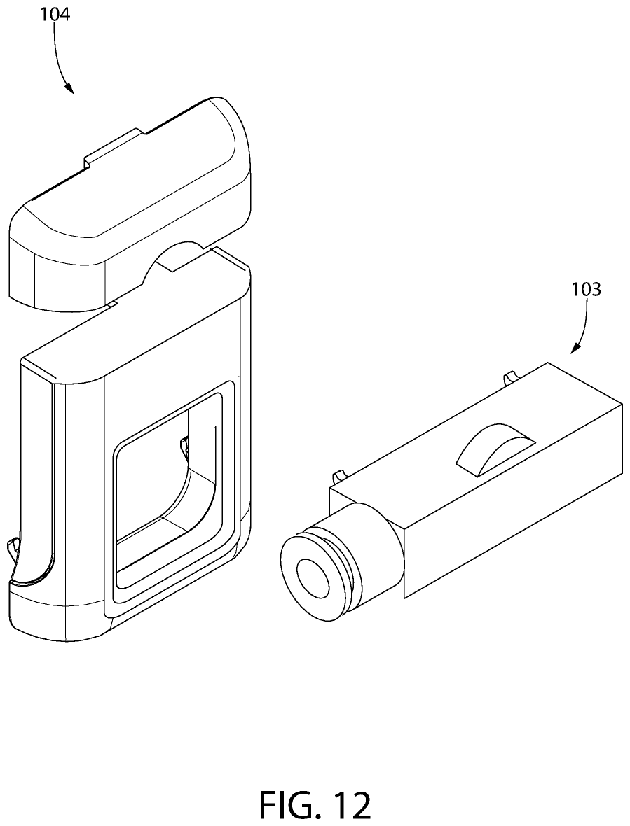

The shapes of the base and cartridge are not essential to the present invention, and alternative embodiments of the device could look different from the views shown in the drawings. Embodiments of the base 102 are shown in FIG. 9 and FIG. 10. Embodiments of the single-use cartridge 103 are shown in FIGS. 10-12. FIG. 12 shows an embodiment of a cartridge 103 comprising a dosing chamber and an intermediate portion 104 of the device to which the cartridge attaches. The intermediate portion 104 may form part of the base or the cartridge. As is evident from FIGS. 10-12, the base and cartridge may vary greatly in terms of their shape. For example, the cartridge 103 may be substantially straight because the air flow conduit extends along a longitudinal axis, as shown in FIG. 12, or the cartridge may have an alternative shape because the air flow conduit is curved, as shown in FIG. 11. The embodiments of the inhaler shown in FIGS. 10-12 are for illustrative purposes only. Alternative embodiments of the device could look different from the exterior views shown in the drawings. For example, FIGS. 3A, 3B and 3C are schematic diagrams of additional embodiments of a medicament delivery device comprising a base 102 and single-use cartridge 103, in which the arrows illustrate the direction of air flow when a subject inhales through the mouthpiece of the cartridge.

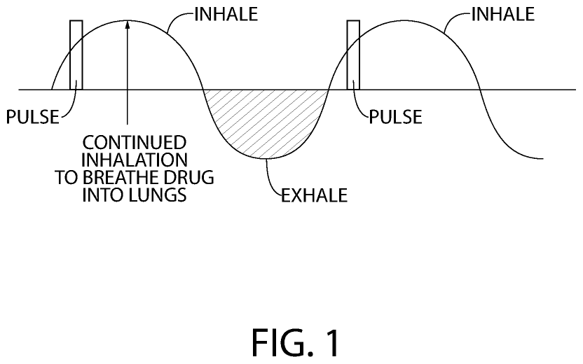

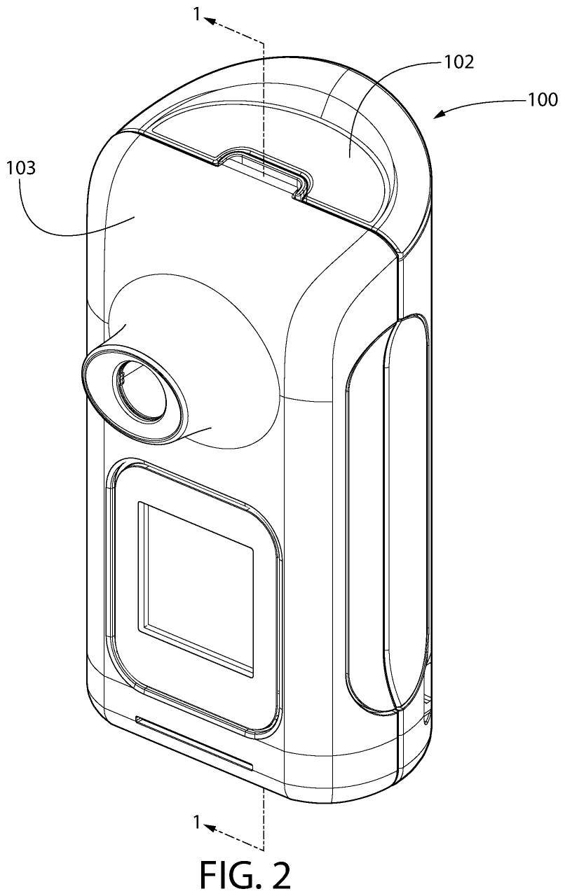

According to exemplary embodiments, the inhaler comprises an inhalation sensor (also referred to herein as a flow sensor or breath sensor) that senses when a patient inhales through the device; for example, the inhalation sensor may be in the form of a pressure sensor, air stream velocity sensor or temperature sensor. Thus, according to one embodiment, the transducer is activated each time a sensor detects an inhalation by a user such that the dose is delivered over several inhalations by the user. The relatively short time period of transducer activation at the beginning of a user's inhalation and the delivery over several inhalations may allow a user to utilize their natural, tidal breathing pattern to receive the pharmaceutical dose as best seen in FIG. 1.

Preferably, the breath sensor is a pressure sensor. Non-limiting examples of pressure sensors that may be used in accordance with embodiments of the present invention include a microelectromechanical system (MEMS) pressure sensor or a nanoelectromechanical system (NEMS) pressure sensor, as described in WO 2016/033418, which is incorporated by reference herein. The inhalation sensor may be located in or near an air flow conduit to detect when a user is inhaling through the mouthpiece in order to trigger the motor to advance a dose. According to a preferred embodiment, the inhaler comprises a pressure sensor pneumatically coupled to an air flow conduit through which the user can inhale; a processor configured to process data received from the sensor to make a determination that inhalation of a breath through the air flow conduit is in progress (or when exhalation is occurring); a controller configured to, responsive to said determination, issue a start dosing signal; and an aerosol engine configured to release dry powder medicament into the air flow conduit during inhalation in response to receiving the start dosing signal.

An aerosol engine preferably refers to an assembly that causes a powder formulation to be aerosolized as it is transferred from a container (e.g., dosing chamber) and entrained in a subject's inhaled air flow. Aerosolizing preferably comprises converting a mass of powder inside a container (e.g., dosing chamber) into particles that are sufficiently deagglomerated (i.e., small and light enough) to be carried in the air.

According to an embodiment, the device is configured to administer dry powder medicament during a dosing breath of an inhalation cycle, preferably over the course of multiple dosing breaths. In one embodiment, during each dosing breath, when the patient inhales through the device and the inhalation sensor detects the inhalation, the aerosol engine is triggered to deliver dry powder medicament to the patient by causing medicament in the drug container (e.g., dosing chamber) to become aerosolized and entrained in the patient's inhaled air. Preferably, the aerosol engine comprises a vibratory element that vibrates upon activation. According to exemplary embodiments, the aerosol engine comprises a vibrating means such as a transducer (e.g., piezoelectric transducer) that confronts a dosing chamber, as described in more detail below. In some embodiments, the inhalation sensor is configured to signal the detection of a dosing breath only after an activation event has occurred. That activation event may include a selected number of breaths (e.g., 1, 2, 3, 4 or 5 preliminary breaths), a fixed quantity of breaths (e.g., a total volume or mass of air is breathed) or a selected threshold is met.

According to an exemplary embodiment, when the inhalation sensor detects a dosing breath, an electrical signal is supplied to a vibratory element that converts the electrical signal into mechanical vibrations and acoustic energy. The vibratory element is preferably a transducer, more preferably a piezoelectric transducer or “piezo.” When the transducer is activated to vibrate, the vibration and resulting acoustic waves cause the dry powder medicament in the container to become aerosolized so that it can be entrained in the patient's inhaled air. According to an embodiment, upon activation of the transducer, mechanical vibration and/or acoustic waves cause at least a portion of the medicament in the dosing chamber to be ejected from one or more openings in the dosing chamber into the air flow conduit so that it becomes entrained in the inhaled breath of the patient. Preferably, the transducer is triggered by each sensed dosing breath in an inhalation cycle to administer at least a portion of the dry powder medicament dose, whereby the dose is administered over a plurality of dosing breaths.

According to a preferred embodiment, a method of using the inhaler comprises completing an inhalation cycle of consecutive inhalations from the inhaler (e.g., from a mouthpiece of the inhaler). As used herein, an inhalation cycle preferably refers to a user's consecutive inhalations through the inhaler in order to receive a dose of medicament. Consecutive inhalations refer to a series of inhalations over which a dose of dry powder medicament is administered by the inhaler, including whether the subject inhales through the inhaler on every inhalation in the series, or whether the subject periodically inhales air that does not contain medicament over the course of the series. Preferably, the subject inhales through the inhaler on every inhalation over the course of the series. Consecutive inhalations may include dosing breaths, which trigger drug delivery, in addition to breaths that do not trigger drug delivery, such as verifying breaths and dose advance breaths.

The inhaler of embodiments of the present invention is capable of administering a dose of medicament in accordance with several possible dosing schemes (i.e., variations of an inhalation cycle), as described below. A dosing scheme may vary according to the number of consecutive inhalations in an inhalation cycle, the number of dosing breaths in an inhalation cycle, the number of times a transducer is activated in an inhalation cycle (which preferably equals the number of dosing breaths in an inhalation cycle), the total amount of on-time that the transducer is activated over an inhalation cycle, and the amount of time that a transducer is activated in response to each dosing breath. The inhaler (e.g., controller) may be programmed with different drive schemes as described herein; for example, the controller may be configured (programmed) to activate the transducer for a total on-time of 5 seconds or less over 2-20 tidal inhalations and/or the controller may be configured (programmed) to activate the transducer for between about 50-1000 milliseconds (ms) during a dosing breath.

Preferably, an inhalation cycle comprises from 2 to 30 consecutive inhalations, or from 2 to 20 consecutive inhalations, or from 3 to 30 consecutive inhalations, or from 3 to 20 consecutive inhalations, or from 2 to 15 consecutive inhalations, or from 3 to 15 consecutive inhalations, or from 2 to 12 consecutive inhalations, or from 3 to 12 consecutive inhalations, or from 2 to 10 consecutive inhalations, or from 3 to 10 consecutive inhalations, or from 2 to 8 consecutive inhalations, or from 3 to 8 consecutive inhalations, or from 4 to 30 consecutive inhalations, or from 4 to 20 consecutive inhalations, or from 4 to 15 consecutive inhalations, or from 4 to 12 consecutive inhalations, or from 4 to 10 consecutive inhalations, or from 4 to 8 consecutive inhalations, or from 5 to 30 consecutive inhalations, or from 5 to 20 consecutive inhalations, or from 5 to 10 consecutive inhalations, or 30 consecutive inhalations or fewer, or 20 consecutive inhalations or fewer, or 15 consecutive inhalations or fewer, or 12 consecutive inhalations or fewer, or 10 consecutive inhalations or fewer, or 8 consecutive inhalations or fewer, or 6 consecutive inhalations or fewer, or 5 consecutive inhalations or fewer. As described in more detail below, the inhalations in an inhalation cycle may include one or more activation events which do not cause the device to administer medicament (e.g., one or more verifying breaths and/or one or more dose advance breaths) in addition to a plurality of the dosing breaths, which cause the device to administer medicament.

Exemplary embodiments of the inhaler provide a short duration of administration because so few inhalations are necessary to deliver a dose, especially when fewer than 30 breaths, fewer than 20 breaths, fewer than 15 breaths, fewer than 12 breaths, fewer than 10 breaths, fewer than 8 breaths, or fewer than 6 breaths are needed; for example, the inhaler is capable of delivering a dose of medicament within 5 minutes or less, or within 4 minutes or less, or within 3 minutes or less, or within 2 minutes or less, or preferably within 90 seconds or less, or within 60 seconds or less, or within 45 seconds or less, or within 30 seconds or less.

According to one embodiment, during the first inhalation in an inhalation cycle, the device verifies that it is an actual breath and not a false trigger, and looks for a second inhalation to validate the inhalation; for example, a processor configured to process data received from the sensor makes a determination that inhalation of a breath through the air flow conduit is in progress. Thus, according to one embodiment, the first breath is a verifying breath. Verifying breaths are optional, and not required in every dosing scheme embodiment. According to another embodiment, at least one inhalation in an inhalation cycle causes the device to advance a dose of medicament into dosing position (referred to as a dose advance breath), such as by advancing the dosing chamber so that it confronts the transducer. Thus, an inhalation cycle may include a dose advance breath.

According to one embodiment, the first breath of an inhalation cycle is a dose advance breath. According to another embodiment, the first breath of an inhalation cycle is a verifying breath. According to an alternative embodiment, the first breath of an inhalation cycle is a verifying breath and the second breath is a dose advance breath. Preferably, medicament is not administered during a verifying breath or dose advance breath. Verifying breaths and dose advance breaths are also referred to herein as activation events because they may activate the device so that it is ready to administer medicament, but preferably do not cause the device to administer medicament. According to an additional embodiment, the dosing scheme does not include any verifying breaths or dose advance breaths.

The inhaler is preferably configured to trigger a vibratory element during each dosing breath of an inhalation cycle in order to administer a dose of dry powder medicament over the course of the inhalation cycle. A portion of a dose of dry powder medicament is preferably administered during each dosing breath, although it is possible that a subject may continue taking one or more dosing breaths after the full dose is delivered, in which case medicament may not be administered, or only a negligible amount may be administered, during the last dosing breath(s) in an inhalation cycle. Consecutive dosing breaths preferably refer to a series of inhalations over which a dose of dry powder medicament is administered by the inhaler, including whether the subject inhales through the inhaler on his or her every inhalation over the course of the series, or whether the subject periodically inhales air that does not contain medicament over the course of the series. Preferably, the subject inhales through the inhaler on his or her every dosing breath over the course of the series.

Preferably, an inhalation cycle comprises from 2 to 30 consecutive dosing breaths, or from 2 to 20 consecutive dosing breaths, or from 2 to 15 consecutive dosing breaths, or from 2 to 12 consecutive dosing breaths, or from 2 to 10 consecutive dosing breaths, or from 2 to 8 consecutive dosing breaths, or from 3 to 30 consecutive dosing breaths, or from 3 to 20 consecutive dosing breaths, or from 3 to 15 consecutive dosing breaths. Most preferably, the inhalation cycle comprises from 3 to 12 consecutive dosing breaths, or from 3 to 10 consecutive dosing breaths, or from 3 to 8 consecutive dosing breaths, or from 4 to 12 consecutive dosing breaths, from 4 to 10 consecutive dosing breaths, or from 4 to 8 consecutive dosing breaths, or from 4 to 6 consecutive dosing breaths, or 30 consecutive dosing breaths or fewer, or 20 consecutive dosing breaths or fewer, or 15 consecutive dosing breaths or fewer, or 12 consecutive dosing breaths or fewer, or 10 consecutive dosing breaths or fewer, or 8 consecutive dosing breaths or fewer, or 6 consecutive dosing breaths or fewer, or 5 consecutive dosing breaths or fewer, or 4 consecutive dosing breaths or fewer, or 3 consecutive dosing breaths or fewer. As described above, the consecutive inhalations in each inhalation cycle may comprise one or more verifying breaths and/or one or more dose advance breaths (i.e., activation events) in addition to the dosing breaths.

According to particular embodiments, feedback may be provided to the patient via one or more indicators, e.g., lights that illuminate during an inhalation cycle (e.g., light-emitting diodes, LEDs) and/or a screen on the device that communicates the status of drug delivery. For example, when inhalation is in progress, a light on the device illuminates a first color (e.g., blue) with each inhalation, confirming that the inhalation sequence progressed correctly, and illuminates a second color that is the same or different from the first color (e.g., green) at the completion of the dose.

As described herein, the inhaler comprises a reusable component or “base” that attaches to a replaceable component or “cartridge,” wherein the replaceable component comprises the one or more doses of medicament, such as a single dose contained inside the dosing chamber. According to one embodiment, the reusable component comprises one or more of a power source (e.g., battery), breath sensor, controller, and transducer; and the replaceable cartridge comprises one or more doses of medicament, dosing chamber, air flow conduit, and mouthpiece. For example, the reusable component may comprise the power source and controller; and the disposable cartridge may comprise the one or more doses of medicament (e.g., one dose contained inside the dosing chamber). Alternative embodiments are also contemplated in which any of the power source, breath sensor, controller, transducer or mouthpiece could form part of the replaceable component instead of the reusable component; and/or any of the dosing chamber or air flow conduit could form part of the reusable component instead of the replaceable component. The reusable component preferably comprises a user interface (e.g., screen display); however, the user interface may alternatively be part of the replaceable component. Also, the replaceable component (cartridge) preferably comprises the air flow conduit; however, the air flow conduit may alternatively be part of the reusable component, or one portion of the air flow conduit may be part of the replaceable component and another portion of the air flow conduit may be part of the reusable component.

According to a preferred method of using the inhaler, the user attaches the cartridge to the base prior to using the device to administer medicament. Thus, a method of using the inhaler may include a step of attaching the base to the cartridge, prior to using the inhaler to administer medicament. For example, the method may comprise steps of attaching the base to the cartridge, turning on the device (e.g., by pressing a button or touch screen on the inhaler, or by another activation event) and inhaling through the device to initiate dosing.

According to a preferred embodiment, the inhaler of the present invention is a handheld device, i.e., it is small enough to be held in a human's hand. This is contrary to conventional nebulizers, which are typically large and bulky, and enable a user to hold only the mouthpiece in his or her hand. For example, the inhaler of the present invention preferably has a width of about 50 mm to about 100 mm, or about 50 mm to about 90 mm, or about 60 mm to about 100 mm, or about 60 mm to about 90 mm, or about 60 mm to about 80 mm; and a height of about 100 mm to about 140 mm, or about 100 mm to about 130 mm, or about 100 mm to about 120 mm, or about 110 mm to about 140 mm, or about 110 mm to about 130 mm, or about 120 mm to about 130 mm; and a depth (excluding the mouthpiece that extends from the surface of the device) of about 50 mm to about 80 mm, or about 50 mm to about 70 mm, or about 50 mm to about 60 mm, or about 60 mm to about 80 mm, or about 60 mm to about 70 mm. For example, the inhaler may have dimensions of about 100-140 mm (height) by about 55-95 mm (width) by about 45-75 mm (depth, excluding the mouthpiece). The mouthpiece may be any size; preferably, the mouthpiece extends about 15 mm to about 70 mm, or about 20 mm to about 70 mm, or about 30 mm to about 70 mm, or about 15 mm to about 60 mm, or about 15 mm to about 50 mm, or about 15 mm to about 40 mm, or about 15 mm to about 30 mm from the surface of the device.

According to a preferred embodiment, the inhaler comprises a controller, i.e., one or more components and associated circuitry integrated into one or more circuit boards for control of the inhaler, data storage and programming interface. Preferably, the inhaler comprises a power source (e.g., a battery, solar cell, etc.) that interfaces with the controller, so that power is provided to the inhaler by the battery. The battery is preferably rechargeable, whereby it can be charged via an external power adapter and allows multiple doses to be administered before requiring recharge. Preferably, the battery is a lithium ion rechargeable battery that provides power for the electronics and excitation of the vibratory element (e.g., piezoelectric transducer). Preferably, the battery meets the following specifications: 0.1-450 mA and voltage 3000-5000 mV, or 3500-4500 mV, or 3700-4300 mV.

According to a preferred embodiment, the inhaler has a flow resistance from about 0.040 cmH₂O^{0.5}/LPM to about 0.12 cmH₂O^{0.5}/LPM, or from about 0.040 cmH₂O^{0.5}/LPM to about 0.1 cmH₂O^{0.5}/LPM, or from about 0.040 cmH₂O^{0.5}/LPM to about 0.095 cmH₂O^{0.5}/LPM, or from about 0.040 cmH₂O^{0.5}/LPM to about 0.090 cmH₂O^{0.5}/LPM, or from about 0.050 cmH₂O^{0.5}/LPM to about 0.12 cmH₂O^{0.5}/LPM, or from about 0.050 cmH₂O^{0.5}/LPM to about 0.1 cmH₂O^{0.5}/LPM, or from about 0.050 cmH₂O^{0.5}/LPM to about 0.095 cmH₂O^{0.5}/LPM, or from about 0.050 cmH₂O^{0.5}/LPM to about 0.090 cmH₂O^{0.5}/LPM, or from about 0.040 cmH₂O^{0.5}/LPM to about 0.085 cmH₂O^{0.5}/LPM, or from about 0.050 cmH₂O^{0.5}/LPM to about 0.085 cmH₂O^{0.5}/LPM, or from about 0.060 cmH₂O^{0.5}/LPM to about 0.085 cmH₂O^{0.5}/LPM, or from about 0.070 cmH₂O^{0.5}/LPM to about 0.085 cmH₂O^{0.5}/LPM, or from about 0.070 cmH₂O^{0.5}/LPM to about 0.09 cmH₂O^{0.5}/LPM, or from about 0.070 cmH₂O^{0.5}/LPM to about 0.095 cmH₂O^{0.5}/LPM, or from about 0.070 cmH₂O^{0.5}/LPM to about 0.1 cmH₂O^{0.5}/LPM, or from about 0.070 cmH₂O^{0.5}/LPM to about 0.12 cmH₂O^{0.5}/LPM at a flow rate of about 30 liters per minute (LPM). Flow resistance may be determined by known methods, such as, the method described in Example 2. Many commercially available inhalers have a flow resistance that is higher than that of the present invention. For most commercially available inhalers with flow resistance similar to the present invention, their optimal performance is typically at a flow rate of 60 L/min or higher, but many children and adult patients with compromised lung function are unable to generate a flow rate of 60 L/min at that level of resistance, and such sub-optimal flow rates may result in incomplete dispersion of the dry powder, an increase of particle size and ultimately lower dosing to the lower airway. As described below, the inhaler of the present invention is capable of delivering therapeutically effective doses of dry powder medicament at flow rates as low as 15 Liters per minute (L/min or LPM), or as low as 20 LPM, or as low as 25 LPM, or as low as 30 LPM while still achieving the preferred APSD profiles described herein (e.g., MMAD, FPF, etc.).

The inhaler of the present invention can be used for the delivery of various types and amounts of dry powder formulations, as the overall dosing schemes (e.g., number of dosing breaths, length of piezo burst per breath, etc.) can be adjusted to deliver a particular dose. The amount of a single dose of dry powder formulation contained inside the inhaler (e.g., the amount of dry powder drug formulation in the dosing chamber) varies depending on several factors, such as the internal volume of the dosing chamber, the density of the formulation, the type and amount of API(s) contained in the formulation, the targeted delivered dose of the API(s), the targeted APSD profile of the formulation, and the type and amount of excipients included in the formulation. The density of the dry powder formulation will vary depending, for example, on whether it is a spray-dried formulation or a micronized formulation; e.g., if a micronized powder has an approximate density of about 0.5 g/cm³, the dosing chamber may hold up to about 250 mg of dry powder formulation, whereas if a spray dried powder has a lower approximate density of about 0.2 g/cm³, the same dosing chamber may hold up to about 100 mg of dry powder formulation.

According to an embodiment, the amount of dry powder formulation in a dose that is contained in the dosing chamber is from about 1 mg to about 250 mg, or from about 1 mg to about 150 mg, or from about 1 mg to about 100 mg, or from about 1 mg to about 90 mg, or from about 1 mg to about 80 mg, or from about 1 mg to about 70 mg, or from about 1 mg to about 60 mg, or from about 1 mg to about 50 mg, or from about 1 mg to about 40 mg, or from about 1 mg to about 30 mg, or from about 1 mg to about 20 mg, or from about 1 mg to about 10 mg, or from about 3 mg to about 250 mg, or from about 3 mg to about 150 mg, or from about 3 mg to about 100 mg, or from about 3 mg to about 90 mg, or from about 3 mg to about 80 mg, or from about 3 mg to about 70 mg, or from about 3 mg to about 60 mg, or from about 3 mg to about 50 mg, or from about 3 mg to about 40 mg, or from about 3 mg to about 30 mg, or from about 3 mg to about 20 mg, or from about 3 mg to about 10 mg, or from about 5 mg to about 250 mg, or from about 5 mg to about 150 mg, or from about 5 mg to about 100 mg, or from about 5 mg to about 90 mg, or from about 5 mg to about 80 mg, or from about 5 mg to about 70 mg, or from about 5 mg to about 60 mg, or from about 5 mg to about 50 mg, or from about 5 mg to about 40 mg, or from about 5 mg to about 30 mg, or from about 5 mg to about 20 mg, or from about 5 mg to about 10 mg, or from about 10 mg to about 250 mg, or from about 10 mg to about 150 mg, or from about 10 mg to about 100 mg, or from about 10 mg to about 90 mg, or from about 10 mg to about 80 mg, or from about 10 mg to about 70 mg, or from about 10 mg to about 60 mg, or from about 10 mg to about 50 mg, or from about 10 mg to about 40 mg, or from about 10 mg to about 30 mg, or from about 10 mg to about 20 mg.

According to an embodiment, the dry powder drug formulation in each dose (e.g., in the dosing chamber) of the present invention comprises at least one medicament and at least one carrier, such as lactose (e.g., lactose monohydrate). For example, the dry powder drug formulation in each dose may comprise at least one medicament in combination with at least 70 wt % carrier (e.g., lactose), or at least 75 wt % carrier, or at least 80 wt % carrier, or at least 85 wt % carrier, or at least 90 wt % carrier, or at least 92 wt % carrier, or at least 95 wt % carrier, or at least 96 wt % carrier, or at least 97 wt % carrier, or at least 97.5 wt % carrier, or at least 98 wt % carrier, or at least 98.5 wt % carrier, or at least 99 wt % carrier, or at least 99.5 wt % carrier, or from 85 wt % to 99.9 wt %, or from 90 wt % to 99.9 wt %, or from 92 wt % to 99.9 wt %, or from 95 wt % to 99.9 wt %, or from 97 wt % to 99.9 wt %, or from 97.5 wt % to 99.9 wt % carrier.

According to one embodiment, the carrier and medicament(s) are blended together by a conventional mixing process, such as high shear mixing; for example, they are not blended by co-spray drying the carrier and medicament(s) together. According to one such embodiment, the lactose has a particle size distribution of approximately the following: D₁₀: 10 micrometers or less; D₅₀: 70 micrometers or less; D₉₀: 200 micrometers or less. According to another embodiment, the lactose has a particle size distribution of approximately the following: D₁₀: 2 micrometers or more; D₅₀: 30 micrometers or more; D₉₀: 120 micrometers or more. According to another embodiment, the lactose has a particle size distribution of approximately the following: D₁₀: 2-10 micrometers; D₅₀: 30-70 micrometers; D₉₀: 120-200 micrometers. According to another embodiment, the lactose has a particle size distribution of approximately the following: D₁₀: 3-7 micrometers; D₅₀: 37-61 micrometers; D₉₀: 124-194 micrometers. According to one embodiment, lactose monohydrate used in the formulation is Respitose® ML001.

According to an alternative embodiment, the carrier(s) and/or excipient(s) are blended with the medicament(s) by co-spraying them together, such as by spray drying. In accordance with this type of embodiment, it may not be necessary or desirable to combine the spray-dried formulation with another carrier such as lactose.

According to particular embodiments, the total amount of the at least one medicament in the drug formulation (e.g., one, two, or three medicaments) is from 0.1 wt % to 80 wt %, or from 0.1 wt % to 70 wt %, or from 0.1 wt % to 60 wt %, or from 0.1 wt % to 50 wt %, or from 0.1 wt % to 40 wt %, or from 0.1 wt % to 35 wt %, or from 0.1 wt % to 30 wt %, or from 0.1 wt % to 25 wt %, or from 0.1 wt % to 20 wt %, or from 0.1 wt % to 15 wt %, or from 0.1 wt % to 12 wt %, or from 0.1 wt % to 10 wt %, or from 0.1 wt % to 8 wt %, or from 0.1 wt % to 6 wt %, or from 0.1 wt % to 5 wt %, or from 0.1 wt % to 4 wt %, or from 0.1 wt % to 3 wt %, or from 0.1 wt % to 2.5 wt %, or from 0.1 wt % to 2 wt %, or from 0.1 wt % to 1.5 wt %, or from 0.1 wt % to 1 wt %. The formulation may optionally comprise one or more excipients, such as magnesium stearate. Examples of API's that may be included in the formulations are described below and in the Examples. According to one embodiment, each drug formulation comprises a LAMA (e.g., glycopyrronium bromide or tiotropium bromide) and/or a LABA (e.g., formoterol fumarate). According to another embodiment, each drug formulation comprises albuterol sulfate. According to another embodiment, each drug formulation comprises a biologic compound, such as DNase (e.g., dornase alfa). According to another embodiment, each drug formulation comprises one or more antibiotics.

According to an embodiment, energy transfer (e.g., in the form of mechanical vibrations and/or acoustic energy) from the vibratory element to the container (e.g., dosing chamber) causes the device to administer the therapeutically effective dose of medicament over the course of the inhalation cycle. According to an embodiment, energy transfer (e.g., in the form of mechanical vibrations and/or acoustic energy) from the vibratory element to a container (e.g., dosing chamber) causes the device to administer at least 75%, or at least 80%, or at least 85%, or at least 90%, or at least 95%, or at least 96%, or at least 97%, or at least 98%, or at least 99%, or 100% of the drug formulation in the dose (e.g., contained inside the dosing chamber) over the course of the inhalation cycle. The percentage of powder left in the dose may be determined, for example, by weighing the container before and after an inhalation cycle and determining the % difference. Preferably, all the dry powder inside the container is administered over the course of an inhalation cycle (with the understanding that a small but consistent amount of powder may still be left in the container after the entire dose is administered; for example, a slight film, or negligible amount, of powder may remain on the surface of the container), or substantially all the contents are administered from the container.

According to preferred embodiments, the inhaler is capable of achieving these levels of clearance from the dosing chamber over a wide range of user flow rates, for example, at flow rates as low as 15 L/min (LPM), or ranging from about 15 L/min to about 90 L/min, or from about 15 L/min to about 60 L/min, or about 15 L/min to about 30 L/min, or about 22 L/min to about 32 L/min, or about 30 L/min to about 60 L/min, or about 30 L/min to about 90 L/min. Thus, according to preferred embodiments, the entire dose contained inside the dosing chamber, or nearly the entire dose, can be administered over the course of an inhalation cycle (e.g., over 5-10 consecutive inhalations, or over 4-8 dosing breaths, etc.) regardless of whether a user that inhales through the device via tidal inhalation or via a strong inhalation, and also regardless of whether the user has compromised lung function.

According to preferred embodiments, when one dose is delivered from each of ten inhalers, each inhaler administers from 65% to 135%, or from 75% to 125%, or from 80% to 120% of the targeted delivered dose and/or the device administers a mean of from 65% to 135%, or from 75% to 125%, or from 80% to 120% of the targeted delivered dose. For example, the device maintains a delivered dose uniformity of ±20% or ±25% or ±35% for each dose across the ten inhalers. Preferably, this delivered dose uniformity is achieved at flow rates as low as 15 L/min (LPM), or ranging from about 15 L/min to about 90 L/min, or from about 15 L/min to about 60 L/min, or from about 15 L/min to about 30 L/min, or about 22 L/min to about 32 L/min, or from about 30 L/min to about 60 L/min, or from about 30 L/min to about 90 L/min or at flow rates of 15 L/min and/or 30 L/min and/or 60 L/min and/or 90 L/min. As used herein, a targeted delivered dose preferably refers to the nominal dose of medicament that is intended to be delivered by the inhaler for purposes of in vitro testing, or the nominal dose of medicament that is prescribed by a physician to be delivered by the inhaler. The targeted delivered dose of medicament is not necessarily the same as the amount of loaded dose that is contained inside the device; for example, a dosing chamber may contain 5 mcg loaded dose of medicament with a targeted delivered dose or nominal dose of 4 mcg. The amount of a dose that is administered or delivered by the inhaler preferably refers to an amount that exits the inhaler and that can be measured by in vitro test methods. The actual amount of drug delivered to a subject's lungs will depend on patient factors, such as anatomical attributes and inspiratory flow profile.

According to preferred embodiments, the inhaler delivers a fine particle fraction (FPF) of at least 30%, or at least 35%, or at least 40%, or at least 45%, or at least 50%, or from about 30% to about 90%, or from about 30% to about 80%, or from about 30% to about 70%, or from about 30% to about 60%, or from about 30% to about 50%, or from about 40% to about 90%, or from about 40% to about 80%, or from about 40% to about 70%, or from about 40% to about 60%. The FPF may vary depending on several factors, such as the density of the formulation, the type and amount of API(s) contained in the formulation (e.g., biologic vs. small molecule) and the type and amount of excipients included in the formulation. As used herein, FPF refers to the percentage of the delivered dose that has an aerodynamic diameter less than or equal to 5 micrometers (μm). Preferably, this FPF is achieved at flow rates as low as 15 L/min, or ranging from about 15 L/min to about 90 L/min, or from about 15 L/min to about 60 L/min, or from about 15 L/min to about 30 L/min, or from about 22 L/min to about 32 L/min, or from about 30 L/min to about 60 L/min, or from about 30 L/min to about 90 L/min or at flow rates of 15 L/min and/or 30 L/min and/or 60 L/min and/or 90 L/min.

According to preferred embodiments, the inhaler of the present invention delivers dry powder medicament comprising particles having a size sufficiently small so as to be delivered to the lungs. For optimal delivery to the lungs, the dry powder preferably should be micronized or spray dried to a mass median aerodynamic diameter powder size of from about 0.1 microns to about 10 microns, preferably from about 0.5 microns to about 6 microns. However, other methods for producing controlled size particles, e.g. supercritical fluid processes, controlled precipitation, etc., also advantageously may be employed. “Mass median aerodynamic diameter” or “MMAD” as used herein preferably refer to the median aerodynamic size of a plurality of particles, typically in a polydisperse population. The “aerodynamic diameter” is preferably the diameter of a unit density sphere having the same settling velocity, generally in air, as a powder and is therefore a useful way to characterize an aerosolized powder or other dispersed particle or particle formulation in terms of its settling behavior. MMAD is determined herein by cascade impaction.

According to preferred embodiments, the inhaler delivers dry powder formulations having an MMAD of about 10 μm (microns) or less, or about 8 microns or less, or about 6 microns or less, or about 5 microns or less, or about 4 μm or less, or about 3.75 microns or less, or about 3.5 microns or less, or about 3.0 microns or less, or from about 0.1 μm to about 10 μm, or from about 0.1 μm to about 8 μm, or from about 0.1 μm to about 6 μm, or from about 0.1 μm to about 5 μm, or from about 0.1 μm to about 4 μm, or from about 1 μm to about 10 μm, or from about 1 μm to about 8 μm, or from about 1 μm to about 6 μm, or from about 1 μm to about 5 μm, or from about 1 μm to about 4 μm. Preferably, this MMAD is achieved at flow rates as low as 15 L/min, ranging from about 15 L/min to about 90 L/min, or from about 15 L/min to about 60 L/min, or from about 15 L/min to about 30 L/min, or from about 22 L/min to about 32 L/min, or from about 30 L/min to about 60 L/min, or from about 30 L/min to about 90 L/min or at flow rates of 15 L/min and/or 30 L/min and/or 60 L/min and/or 90 L/min. The MMAD may vary depending on several factors, such as the density of the formulation, the type and amount of API(s) contained in the formulation (e.g., biologic vs. small molecule), the targeted deliver dose of the API(s), the targeted APSD profile of the formulation, and the type and amount of excipients included in the formulation.

According to a preferred embodiment, the inhaler's vibratory element is a piezoelectric transducer, embodiments of which are described in more detail below. According to one embodiment, the amount of voltage supplied to the vibratory element (e.g., piezoelectric transducer) when it is activated to vibrate is from about 180-260 V p-p, or about 190-250 V p-p, or preferably about 200-240 V p-p. According to one embodiment, the piezoelectric transducer is vibrated at a frequency from about 36 kHz to about 43 kHz, or about 37 kHz to about 43 kHz, or about 38 kHz to about 43 kHz, or about 36 kHz to about 42 kHz, or about 36 kHz to about 41 kHz, or about 36 kHz to about 40 kHz, or about 36 kHz to about 39 kHz, or about 37 kHz to about 42 kHz, or about 37 kHz to about 41 kHz, or about 37 kHz to about 40 kHz, or about 38 kHz to about 42 kHz, or about 38 kHz to about 41 kHz, or about 38 kHz to about 40 kHz, or about 38 kHz to about 39 kHz.

According to one embodiment, upon activation by a dosing breath, the piezoelectric transducer (piezo) is activated to vibrate for between about 50 ms to about 1000 ms upon each inhalation. Each activation of the piezo in response to a dosing breath may be referred to as a “burst” or “pulse.” Preferably, this activation or burst is effective to aerosolize at least a portion of a dose toward the beginning of a user's inhalation so that the remainder of the inhalation is chase air that draws the aerosolized dose (or portion thereof) into the user's lungs. According to particular embodiments, the piezoelectric transducer is activated to vibrate for from about 50 ms to about 1000 ms, or from about 50 ms to about 900 ms, or about 50 ms to about 800 ms, about 50 ms to about 700 ms, or about 50 ms to about 600 ms, or about 50 ms to about 500 ms, or about 50 ms to about 400 ms, or about 50 ms to about 300 ms, or about 50 ms to about 200 ms, or about 50 ms to about 100 ms, or about 100 ms to about 900 ms, or about 100 ms to about 800 ms, or about 100 ms to about 700 ms, or about 100 ms to about 600 ms, or about 100 ms to about 500 ms, or about 100 ms to about 400 ms, or about 100 ms to about 300 ms, or about 100 ms to about 200 ms upon each dosing breath.

In accordance with different dosing scheme embodiments, the piezo may be activated for different amounts of time over the course of an inhalation cycle, or may be activated for the same amount of time over the course of an inhalation cycle. For example, the piezo may be activated for 100 ms for each of the first four dosing breaths, and 300 ms for each of the subsequent four dosing breaths over the course of eight total dosing breaths in an inhalation cycle (for a total of 1.6 seconds of “on-time”). According to another example, the piezo may be activated for 500 ms for each of four or five total dosing breaths in an inhalation cycle (for a total “on-time” of 2 seconds or 2.5 seconds, respectively). In one embodiment, the transducer is activated for between about 100 milliseconds to about 500 milliseconds during the first burst of a series of bursts (e.g., from 3 bursts to 12 bursts, or from 3 bursts to 10 bursts, or from 3 bursts to 8 bursts, or from 3 bursts to 6 bursts) to deliver the contents of the dose over the course of the series.

The “on-time” preferably refers to the total amount of time the transducer is activated at its resonant frequency, preferably sufficient to cause synthetic jetting in the dosing chamber, over the course of an inhalation cycle, i.e., the number of bursts that occur at a resonant frequency of the transducer sufficient to cause synthetic jetting (e.g., 4 bursts), multiplied by the amount of time per burst (e.g., 500 ms), over the inhalation cycle (4×500 ms=2 seconds on-time). For example, if a transducer having a resonant frequency between 38-42 kHz is activated a total of 4 times at that frequency for 500 ms each time because the inhalation cycle includes 4 dosing breaths, and each of those activations occurs at a resonant frequency of the transducer sufficient to generate synthetic jetting, the total on-time for that inhalation cycle is 2 seconds (with brief interruptions by hop frequencies included in the on-time, as described herein). An “off-time” is not part of the on-time and preferably includes those periods of time during an inhalation cycle when the transducer is not being activated, or the transducer is activated at one or more frequencies that do not cause the dosing chamber to resonate sufficient to cause synthetic jetting (e.g., the transducer that is resonant at 38-42 kHz runs at a frequency of 10 kHz in between dosing breaths, for a total of 20-30 seconds of off-time over the course of the inhalation cycle), and those “off-time” periods of activation are not considered to be part of the on-time.

According to an embodiment, the transducer is activated to vibrate for a total of 5 seconds or less “on-time” over the course of an inhalation cycle (according to any dosing scheme, e.g., 10 bursts at 500 ms each, etc.), or for a total of 4 seconds or less, or for a total of 3 seconds or less, or for a total of 2 seconds or less, or for a total of from about 1 second to about 5 seconds, or about 1 second to about 4 seconds, or about 1 second to about 3 seconds, or about 1 second to about 2 seconds, or about 1 second to about 1.8 seconds, or about 1 second to about 1.6 seconds, or about 1 second to about 1.4 seconds, or about 1.2 seconds to about 3 seconds, or about 1.2 seconds to about 2 seconds.

According to one embodiment, the aerosol engine is capable of delivering the therapeutically effective dose the course of an inhalation cycle comprising at least three piezo bursts at its resonant frequency, or over at least four piezo bursts, or over at least five piezo bursts, or over at least six piezo bursts, or over at least seven piezo bursts, or over at least eight piezo bursts, or over at least nine piezo bursts, or over at least ten piezo bursts, when the piezo is activated to vibrate for a total on-time of 5 seconds or less over the course of an inhalation cycle, as set forth above. For example, an inhalation cycle may comprise from 3 to 12 piezo bursts at its resonant frequency, or from 3 to 10 piezo bursts, or from 3 to 8 piezo bursts, or from 4 to 12 piezo bursts, from 4 to 10 piezo bursts, or from 4 to 8 piezo bursts, or from 4 to 6 piezo bursts, or 30 piezo bursts or fewer, or 20 piezo bursts or fewer, or 15 piezo bursts or fewer, or 12 piezo bursts or fewer, or 10 piezo bursts or fewer, or 8 piezo bursts or fewer, or 6 piezo bursts or fewer, or 5 piezo bursts or fewer, or 4 piezo bursts or fewer, or 3 piezo bursts or fewer. As described herein, each piezo burst preferably occurs in response to a dosing breath.

According to an embodiment, the medicament delivery device delivers at least 0.1 micrograms (m) of API per piezo activation (burst), or at least 0.5 μg API per burst, or at least 1 μg API per burst, or at least 2 μg API per burst, or at least 3 μg API per burst, or at least 4 μg API per burst, or at least 5 μg API per burst, or at least 6 μg API per burst, or at least 7 μg API per burst, or at least 8 μg API per burst. The amount of API delivered per burst may vary depending on the amount or wt % of API in the dose. The medicament delivery device may deliver different amounts of API per burst over the course of an inhalation cycle; for example, the amount of API delivered by the first burst, or the first two bursts, may be higher than the amount of API delivered by the last burst, or the last two bursts, respectively. In one embodiment, a burst (e.g., the first burst in response to a first dosing breath) delivers at least about 20%, or at least about 30%, or at least about 40%, or at least about 50%, or at least about 60% of the dose.

The amount of dry powder formulation that is delivered in response to each piezo burst will vary depending on several factors, such as the shape of the flow channel (as described in more detail below), the amount of formulation in a dose, and the type and amount of excipients included in the formulation. The medicament delivery device may deliver different amounts of dry powder formulation per burst over the course of an inhalation cycle; for example, the amount of dry powder formulation delivered by the first burst, or the first two bursts, may be higher than the amount of dry powder formulation delivered by the last burst, or the last two bursts, respectively. Preferably, less than 15 mg dry powder formulation, more preferably 10 mg or less dry powder formulation are delivered by a single burst in a dosing scheme, in order to decrease the likelihood that the subject coughs due to inhalation of a high amount of dry powder. For example, the medicament delivery device may deliver between from about 0.1 mg to about 10 mg, or from about 0.1 mg to about 9 mg, or from about 0.1 mg to about 9 mg, or from about 0.1 mg to about 8 mg, or from about 0.1 mg to about 7 mg, or from about 0.1 mg to about 6 mg, or from about 0.1 mg to about 5 mg, or from about 0.1 mg to about 4 mg, or from about 0.1 mg to about 3 mg, or from about 0.1 mg to about 2 mg, or from about 0.1 mg to about 1 mg, or from about 0.1 mg to about 0.5 mg, from about 1 mg to about 10 mg, or from about 1 mg to about 9 mg, or from about 1 mg to about 9 mg, or from about 1 mg to about 8 mg, or from about 1 mg to about 7 mg, or from about 1 mg to about 6 mg, or from about 1 mg to about 5 mg, or from about 1 mg to about 4 mg, or from about 1 mg to about 3 mg, or from about 1 mg to about 2 mg dry powder formulation per piezo burst.

In the example of a dry powder drug formulation comprising at least one API in combination with at least 90 wt % carrier (e.g., lactose), or at least 92 wt % carrier, or at least 95 wt % carrier, or at least 96 wt % carrier, or at least 97 wt % carrier, or at least 97.5 wt % carrier, or at least 98 wt % carrier, or at least 98.5 wt % carrier, or at least 99 wt % carrier, or at least 99.5 wt % carrier, or from 85 wt % to 99.9 wt %, or from 90 wt % to 99.9 wt %, or from 92 wt % to 99.9 wt %, or from 95 wt % to 99.9 wt %, or from 97 wt % to 99.9 wt %, or from 97.5 wt % to 99.9 wt % carrier, in one embodiment, the first burst delivers at least 0.5 micrograms of the API, or at least 1 micrograms of the API, or at least 1.5 micrograms of the API, or at least 2 micrograms of the API, or at least 3 micrograms of the API, or at least 4 micrograms of the API, or at least 5 micrograms of the API, or at least 6 micrograms of the API, or at least 7 micrograms of the API, or at least 8 micrograms of the API, or from about 0.5 micrograms to about 8 micrograms, or from about 0.5 micrograms to about 6 micrograms, or from about 0.5 micrograms to about 4 micrograms of API.

According to one embodiment, the medicament delivery device administers at least about 10%, or at least about 15%, or at least about 20%, or at least about 25%, or at least about 30%, or at least about 40%, or at least about 50%, or at least about 60% of the dose of medicament in response to the first dosing breath (i.e., on the first burst), and the remainder of the dose is administered over the remaining dosing breaths in the inhalation cycle. Stated another way, the medicament delivery device may be configured to administer at least about 10%, or at least about 15%, or at least about 20%, or at least about 25%, or at least about 30%, or at least about 40%, or at least about 50%, or at least about 60% of the dry powder medicament dose in response to a first dosing breath in an inhalation cycle.

In the example of a dry powder drug formulation comprising at least one API in combination with at least 90 wt % carrier (e.g., lactose), or at least 92 wt % carrier, or at least 95 wt % carrier, or at least 96 wt % carrier, or at least 97 wt % carrier, or at least 97.5 wt % carrier, or at least 98 wt % carrier, or at least 98.5 wt % carrier, or at least 99 wt % carrier, or at least 99.5 wt % carrier, or from 85 wt % to 99.9 wt %, or from 90 wt % to 99.9 wt %, or from 92 wt % to 99.9 wt %, or from 95 wt % to 99.9 wt %, or from 97 wt % to 99.9 wt %, or from 97.5 wt % to 99.9 wt % carrier, in one embodiment, the transducer is activated four times from about 400 milliseconds to about 600 milliseconds each time to deliver the complete pharmaceutical dose. The first burst may be configured to deliver about 70% to about 80% of the dose. The second, third, and fourth burst may each be configured to deliver about 5% to about 15% of the dose.

In one embodiment, the first burst delivers at least about 20%, or at least about 30%, or at least about 40%, or at least about 50%, or at least about 60% of the targeted delivered dose of medicament; or from about 40% to about 85% of the targeted delivered dose. According to another embodiment, the first burst delivers at least about 20%, or at least about 30%, or at least about 40%, or at least about 50%, or at least about 60% of the medicament in the dose. In one embodiment, the second burst delivers at least about 5%, or at least about 10%, or at least about 20% of the dose. In one embodiment, the third and fourth bursts each deliver at least about 1%, or at least about 5%, or at least about 10% of the dose. In one embodiment, the remaining bursts deliver the remainder of the original medicament dose.

According to an embodiment, as described in more detail below, upon each activation of the piezoelectric transducer, at least a portion of the dry powder medicament dose aerosolizes, whereby the acoustic waves cause the medicament to be ejected from one or more openings in the dosing chamber into the air flow conduit so that it becomes entrained in the inhaled breath of the patient. Preferably, the inhaler of the present invention employs synthetic jetting to help aerosolize the drug powder. Synthetic jetting has been described in U.S. Pat. Nos. 7,318,434; 7,779,837; 7,334,577; and 8,322,338, which are incorporated by reference herein. As described in the aforesaid patents, if a chamber is bound on one end by an acoustic wave generating device and on the other end by a rigid wall with a small orifice, when acoustic waves are emitted at high enough frequency and amplitude from the generator, a jet of air that emanates from the orifice outward from the chamber can be produced. The jet, or so-called synthetic jet, is comprised of a train of vortical air puffs that are formed at the orifice.

According to particular embodiments, the piezo confronts a dosing chamber, and is capable of achieving maximum synthetic jetting out of the opening(s) in the dosing chamber when the piezo is activated for as few as 50 ms, or as few as 100 ms in a single burst, or as few as 200 ms in a single burst, or as few as 300 ms, or as few as 500 ms in a single burst. Preferably, the synthetic jetting achieves at least 0.5 V, or at least 0.6 V, or at least 0.7 V, or at least 0.8 V, or at least 0.9 V, or at least 1.0 V, or at least 1.1 V, or at least 1.2 V, or at least 1.3 V, or at least 1.4 V, or at least 1.5 V, or at least 1.6 V, or at least 1.7 V; for example, from 0.5 V to 1.7 V, or 0.5 V to 1.6 V, or 0.5 V to 1.5 V, or 0.5 V to 1.4 V, or 0.5 V to 1.3 V, or 0.5 V to 1.2 V, or 0.5 V to 1.0 V, e.g., as quantified by an oscilloscope which converts pressure signals into voltages. Synthetic jetting may be observed and quantified in accordance with the procedure described in Example 1. As described in Example 1, the aerosol engine is connected to a Pneumotach Amplifier 1 (PA-1), which measures gas flow coming out of the dosing chamber opening(s). A differential pressure signal is measured and amplified to provide an analog output proportional to the flow rate. The PA-1 is connected to an oscilloscope, which converts the signal to voltages.

According to preferred embodiments, the inhaler of the present invention is capable of delivering therapeutically effective amounts of dry powder medicament(s) to a subject's lungs, preferably for the treatment of a respiratory disease or disorder, or one more symptoms thereof (e.g., selected from the group comprising or consisting of COPD, asthma, cystic fibrosis, IPF, etc.), preferably when the subject inhales through the inhaler using tidal inhalation. The inhaler is capable of delivering such therapeutically effective amounts within 80% to 120% of a mean delivered dose across a wide range of flow rates (e.g., 15-90 LPM or 15-60 LPM or 30-90 LPM or 30-60 LPM), and preferably across a wide range of transducer drive schemes, wherein the drive schemes vary by the number of bursts (e.g., 4-8 bursts) and the amount of “on-time” per burst (e.g., 100 ms/burst to 500 ms/burst), e.g., for a total “on-time” ranging from about 1 second to about 5 seconds over all the bursts.

The device preferably maintains a consistent aerodynamic particle size distribution (APSD) across different flow rates and preferably across different drive schemes, wherein the mass median aerodynamic diameter (MMAD) is consistently about 10 μm (microns) or less, or about 8 microns or less, or more preferably about 6 microns or less, or about 5 microns or less, or about 4 μm or less, or about 3.75 microns or less, or about 3.5 microns or less, or about 3.0 microns or less, or from about 0.1 μm to about 10 μm, or from about 0.1 μm to about 8 μm, or from about 0.1 μm to about 6 μm, or from about 0.1 μm to about 5 μm, or from about 0.1 μm to about 4 μm, or from about 1 μm to about 10 μm, or from about 1 μm to about 8 μm, or from about 1 μm to about 6 μm, or from about 1 μm to about 5 μm, or from about 1 μm to about 4 μm. Preferably, the FPF is also consistent across different flow rates and drive schemes, e.g., at least 30%, or at least 35%, or at least 40%, or at least 45%, or at least 50%, or from about 30% to about 90%, or from about 30% to about 80%, or from about 30% to about 70%, or from about 30% to about 60%, or from about 30% to about 50%, or from about 40% to about 90%, or from about 40% to about 80%, or from about 40% to about 70%, or from about 40% to about 60%.

The single-dose cartridge inhaler described herein has demonstrated consistent aerosol performance with dry powder formulations that are either micronized or spray-dried, and that contain either small molecule API(s) or large biologic API(s). For example, aerosol performance has been consistent with respect to FPF and MMAD (see, e.g., Examples 3 and 4). In one powder clearance study, for example, the device showed successful delivery of spray dried formulations of antibiotics up to 80 mg when programmed to activate the piezo for 8 timed bursts of 500 ms duration at a flow rate of 30 LPM (small molecule spray dried with a single excipient, ≥90% w/w). The clearance rate can be adjusted via control of electronic and physical parameters of the dosing engine such as: transducer pulse length (piezo on-time), flow channel cross section, and dosing chamber holes and orientation.

According to one embodiment, a medicament delivery device comprises a dosing chamber comprising an interior that is configured to contain dry powder medicament, a transducer confronting the dosing chamber, wherein the dosing chamber and the transducer are acoustically resonant such that the dosing chamber is configured to resonate in response to an activation of the transducer, and a controller electrically coupled to the transducer and configured to send an electrical signal that activates the transducer when the medicament delivery device senses a subject's dosing breath. The medicament delivery device preferably has a flow resistance in a range from about 0.040 cmH₂O^{0.5}/LPM to about 0.1 cmH₂O^{0.5}/LPM, more preferably from about 0.040 cmH₂O^{0.5}/LPM to about 0.090 cmH₂O^{0.5}/LPM, at 30 liters per minute (LPM). The device is preferably capable of delivering a therapeutically effective dose of dry powder medicament in response to between 2-20 tidal inhalations. The device preferably comprises a base and a removable single-dose cartridge, wherein the dosing chamber is contained within the removable single-dose cartridge, and the dose of dry powder medicament is contained inside the dosing chamber prior to delivery of the dose via activation of the transducer.

According to another embodiment, a method of treating a respiratory disease or condition (e.g., COPD, asthma, CF, IPF, etc.), or one or more symptoms thereof (e.g., a method of increasing a subject's FEV₁) comprises inhaling a therapeutically effective dose of dry powder medicament through a medicament delivery device using between 2-20 tidal inhalations over the course of an inhalation cycle, the inhalation cycle comprising dosing breaths, wherein the medicament delivery device comprises a vibratory element that is activated upon each dosing breath and causes dry powder medicament to be aerosolized within a dosing chamber and expelled from one or more openings in the dosing chamber into an air flow conduit, wherein pressure oscillations in the dosing chamber are sufficiently high at the one or more openings to aerosolize and expel the dry powder medicament via synthetic jetting, wherein the medicament delivery device comprises a base and a removable single-dose cartridge, the dosing chamber is contained within the removable single-dose cartridge, and the dose of dry powder medicament is contained inside the dosing chamber prior to the delivery of the dose via activation of the transducer. The medicament delivery device preferably has a flow resistance in a range from about 0.040 cmH₂O^{0.5}/LPM to about 0.1 cmH₂O^{0.5}/LPM, more preferably from about 0.040 cmH₂O^{0.5}/LPM to about 0.090 cmH₂O^{0.5}/LPM, at 30 liters per minute (LPM) and is capable of delivering the dose of dry powder medicament in response to tidal inhalation (e.g., in response to flow rates at least within a range of about 15 LPM to about 30 LPM). Preferably, the dose of dry powder medicament delivered by the medicament delivery device has a mass median aerodynamic diameter (MMAD) of about 6 microns or less and a fine particle fraction of at least 30%. Preferably, the dose of medicament is delivered within 5 minutes or less, or within 4 minutes or less, or within 3 minutes or less, or within 2 minutes or less, or preferably within 90 seconds or less, or within 60 seconds or less, or within 45 seconds or less, or within 30 seconds or less. Preferably, the medicament delivery device is configured to administer at least about 10%, or at least about 15%, or at least about 20%, or at least about 25%, or at least about 30%, or at least about 40%, or at least about 50%, or at least about 60% of the dry powder medicament dose in response to a first dosing breath in an inhalation cycle.

According to another embodiment, a method of treating a respiratory disease or condition (e.g., COPD, asthma, CF, IPF, etc.), or one or more symptoms thereof (e.g., a method of increasing a subject's FEV₁) comprises inhaling a therapeutically effective dose of dry powder medicament through a medicament delivery device using between 2-20 tidal inhalations over the course of an inhalation cycle, the inhalation cycle comprising dosing breaths, wherein the medicament delivery device comprises a vibratory element that is activated upon each dosing breath and causes dry powder medicament to be aerosolized within a dosing chamber and expelled from one or more openings in the dosing chamber into an air flow conduit, wherein pressure oscillations in the dosing chamber are sufficiently high at the one or more openings to aerosolize and expel the dry powder medicament via synthetic jetting, wherein the medicament delivery device comprises a base and a removable single-dose cartridge, the dosing chamber is contained within the removable single-dose cartridge, and the dose of dry powder medicament is contained inside the dosing chamber prior to delivery of the dose via activation of the transducer. The medicament delivery device may comprise a high-velocity flow channel, the high-velocity flow channel comprising a constricted section disposed over the one or more openings in the dosing chamber, or adjacent to the one or more openings in the dosing chamber, wherein the dose of medicament is expelled over the course of the inhalation cycle by a combination of active delivery and passive delivery, and wherein the medicament delivery device is capable of delivering the dose of dry powder medicament in response to tidal inhalation (e.g., in response to flow rates at least within a range of about 15 LPM to about 30 LPM). Preferably, the medicament delivery device has a flow resistance in a range from about 0.040 cmH₂O^{0.5}/LPM to about 0.1 cmH₂O^{0.5}/LPM, more preferably from about 0.040 cmH₂O^{0.5}/LPM to about 0.090 cmH₂O^{0.5}/LPM, at 30 liters per minute (LPM). Preferably, the dose of dry powder medicament delivered by the medicament delivery device has a mass median aerodynamic diameter (MMAD) of about 6 microns or less and a fine particle fraction of at least 30%. Preferably, the dose of medicament is delivered within 5 minutes or less, or within 4 minutes or less, or within 3 minutes or less, or within 2 minutes or less, or preferably within 90 seconds or less, or within 60 seconds or less, or within 45 seconds or less, or within 30 seconds or less.

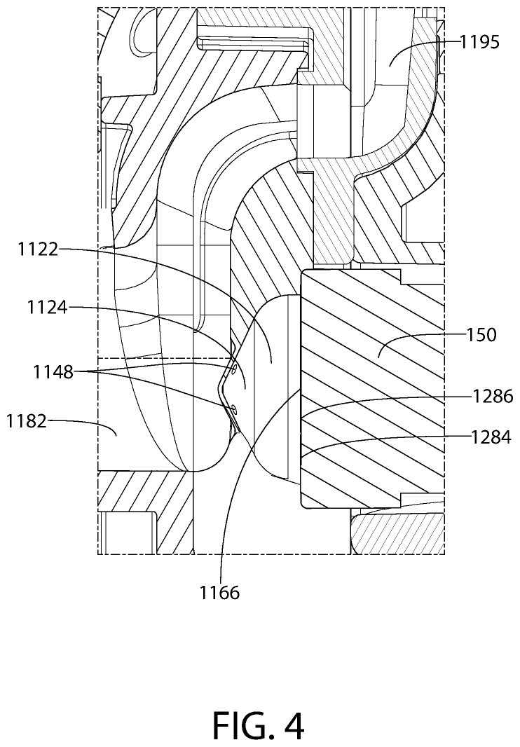

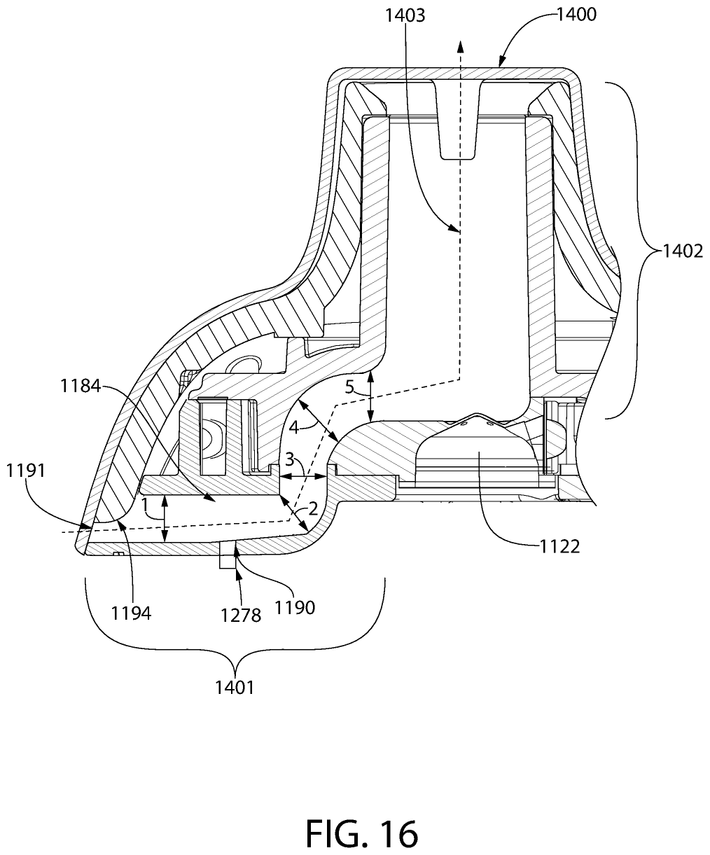

Turning to FIG. 4, in one embodiment, the inhaler 100 includes a channeling means (e.g., air flow conduit 1195) configured to allow air to travel through the inhaler 100 when a user inhales through a mouthpiece. In one embodiment, the inhaler 100 includes a sensor 1278 (see FIG. 16) configured to detect airflow through the air flow conduit 1195 and send a signal to a controller when airflow is detected. A membrane is configured to cover an open end of the dosing chamber 1122 in one embodiment. In one embodiment, a transducer 150 confronts the membrane 1166. In some embodiments, a separation means (e.g., spacer 1286) for separating a vibration means from membrane 1166 is positioned between the transducer 150 and the membrane 1166. In one embodiment, the controller is configured to activate a transducer 150 when an activation event is detected. In one embodiment, detection of multiple inhalations are required to trigger activation of transducer 150. For example, the controller may be configured to activate a transducer 150 when a flow of air is detected by the sensor 1278 (in some cases, when a subsequent flow of air is detected, e.g., second, third, or later). The transducer 150 is configured to vibrate, thereby vibrating the membrane 1166, to aerosolize and transfer drug from the dosing chamber 1122 to the air flow conduit. In one embodiment, the vibration of the transducer 150 delivers the aerosolized pharmaceutical through openings 1148 in the dosing chamber 1122, through the exit channel 1182, and to a user, as explained in greater detail below. In one embodiment, the transducer 150 is configured to transfer acoustic vibration to the membrane. In some embodiments, the transducer 150 is configured to transfer vibration to the membrane 1166 via acoustic vibration and/or physical vibration. In one embodiment, one or more elements (e.g., dosing chamber, transducer, membrane) are configured for efficient energy coupling through a common resonant frequency and/or acoustic impedance matching, as explained in greater detail below.

Embodiments of the present invention relate to a dosing chamber, which may be a molded acoustic chamber designed for ultrasonic synthetic jetting. According to preferred embodiments, the shape of the chamber has been optimized for powder delivery via synthetic jetting. Preferably, precision molding is used to provide a thin chamber top and one or more small jetting holes, i.e., openings that extend through the chamber wall.

According to preferred embodiments, the design of the dosing chamber helps to achieve the following objectives: sufficient volume to allow synthetic jet drug delivery while having a resonance frequency that matches that of a commercially available piezoelectric transducer; achievement of synthetic jetting from the acoustic chamber while providing a sufficient egress area to deliver drug quickly; and sufficient redundancy to prevent loss of delivery function due to intermittent clogging of holes.

In preferred embodiments, the geometries of the dosing chamber are configured such that the dosing chamber would resonate at the same or similar frequencies as the piezoelectric transducer (e.g., from about 37 kHz to about 42 kHz). The chamber geometry may also be configured to provide strong synthetic jetting and uniform dose delivery. As discussed in greater detail below, acoustic resonance frequency is preferably adjusted to match the mechanical resonance frequency of the piezoelectric transducer; that is, to match the frequency at which the maximum real power is consumed, and therefore the desired mechanical displacement is achieved.

Preferably, the geometry, size, and hole placement of the dosing chamber enable the chamber to resonate at a specific frequency coincident with the resonance frequency of the piezoelectric transducer in order to provide fast-onset synthetic jetting and maximal robustness against the effects of temperature, which tends to move the resonance frequencies of the piezoelectric transducer and the acoustic chamber in opposite directions.

According to one embodiment, the inhaler comprises: a dosing chamber containing medicament; a transducer confronting the dosing chamber, the transducer being configured to aerosolize the medicament when the transducer is activated; and a membrane between the dosing chamber and the transducer, the membrane being affixed to the dosing chamber, wherein the inhaler produces a synthetic jet to deliver the aerosolized medicament to a user when the transducer is activated.

According to one embodiment, the geometry, size and hole placement of the dosing chamber are configured such that the inhaler produces synthetic jetting to deliver the aerosolized medicament to a user when the transducer is activated, wherein the synthetic jetting causes medicament to be expelled into the exit channel in response to an activation of the transducer (i.e., a burst of the transducer) as short as 100 milliseconds (e.g., from about 100 ms to about 1000 ms, or from about 100 ms to about 800 ms, or from about 100 ms to about 500 ms).

According to an embodiment, the medicament delivery device comprises a dosing chamber comprising an interior that is configured to contain dry powder medicament and a transducer confronting the dosing chamber. The dosing chamber and transducer are acoustically resonant such that the dosing chamber resonates in response to an activation of the transducer. The dosing chamber has an interior shape, internal height and location of one or more openings configured to cause the dry powder medicament to become aerosolized and delivered from the dosing chamber via synthetic jetting upon an activation of the transducer. Preferably, the dosing chamber's interior shape is at least partially defined by a lower sidewall that transitions to a shoulder, the shoulder transitions to an apex extending away from the lower sidewall, and the apex converges to a point, wherein the one or more openings in the dosing chamber are disposed in the apex. The internal height of the dosing chamber is configured so that pressure oscillation (e.g., at one or more anti-nodes) is sufficiently high to cause the dry powder medicament to become aerosolized and delivered from the one or more openings. Preferably the one or more openings are disposed at one or more anti-nodes of the dosing chamber when the transducer is activated.

According to this embodiment, each of the dosing chamber's (1) interior shape, (2) internal height and (3) location of one or more openings affects the deaggregation and/or delivery of powder. For example, one or more of the speed of onset of synthetic jetting, maximum synthetic jetting, delivered dose per burst, total delivered dose, and aerodynamic particle size distribution may be affected by a change in one or more of the dosing chamber's interior shape, internal height and location of one or more openings. Preferably, the dosing chamber's interior shape and height are configured such that the combined acoustic resonance of the transducer and dosing chamber is sufficient to cause aerosolization and delivery of the dry powder medicament having an MMAD within the preferred ranges described herein, e.g., about 6 μm or less, preferably with a fine particle fraction within the preferred ranges described herein, e.g., at least 30%. Maximum synthetic jetting is preferably achieved within ranges of time described herein, e.g., within about 500 ms or less from the start of a transducer activation.

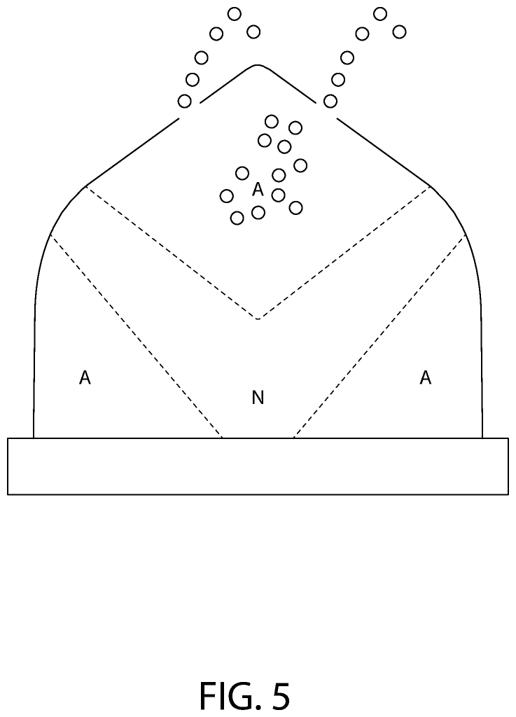

As illustrated in the embodiment shown in FIG. 5, certain areas inside the dosing chamber (labeled “N” for node) exhibit little or no oscillation in pressure when the transducer is activated, whereas other areas (labeled “A” for anti-node) exhibit higher oscillations in pressure when a transducer is activated. The highest amount of synthetic jetting within the dosing chamber, i.e., the occurrence of internal jets that stir the contents of the dosing chamber, occurs in those areas where there is high pressure oscillations, whereas synthetic jetting does not occur (or minimally occurs) in those areas with no oscillating pressure or very little oscillating pressure. Stated another way, the anti-nodes exhibit higher oscillations in pressure relative to the nodes. The opening(s) in the dosing chamber are preferably placed in one or more areas of high oscillating pressure (“anti-nodes”), instead of areas of little or no oscillating pressure (“nodes”), so that synthetic jetting can be maximized at the opening(s). Preferably, the dosing chamber shape, including its conical configuration near the holes, prevents powder from getting into nodes and achieves suitable intensity of oscillating pressures at the anti-nodes. According to a preferred embodiment, optimal synthetic jetting occurs when the dosing chamber's opening(s) are positioned at the conical configuration in the area of an anti-node where there are higher oscillations in pressure compared to the nodes. The location of nodes and anti-nodes inside a chamber can be determined by conventional methods of eigen frequency analysis, based on the size and shape of the chamber (e.g., using Comsol® software).

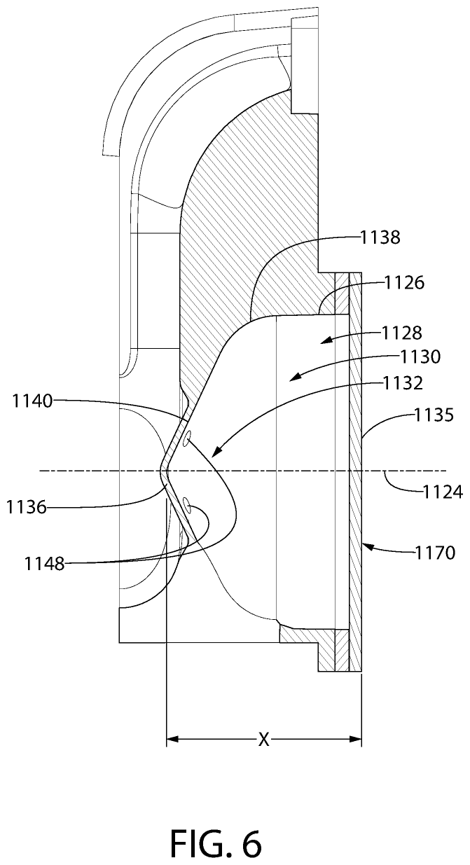

For a transducer's frequency range (e.g., 37-42 kHz), not all internal heights of a dosing chamber will provide suitable synthetic jetting, dose delivery and aerodynamic particle size distribution (APSD) because the internal height affects the acoustic resonance of the system, including the location of nodes and anti-nodes. In some cases, if the internal height of the dosing chamber is changed, the transducer's activation frequency must also be changed, in order to match the new acoustic resonance of the new dosing chamber shape. In other cases, the transducer's activation frequency may remain the same for different internal heights if those heights provide sufficiently high oscillating pressure at the opening(s). According to an embodiment, a dosing chamber having an internal height X, as shown in FIG. 6, has a resonant frequency Y that is approximately the same as that of the transducer; and a dosing chamber having an internal height that is approximately 2X, or between 1.7X and 2.3X (i.e., at the next approximate harmonic) has approximately the same resonant frequency Y because at the next approximate harmonic the anti-nodes (high oscillating pressure) are again located at the opening(s) of the dosing chamber.

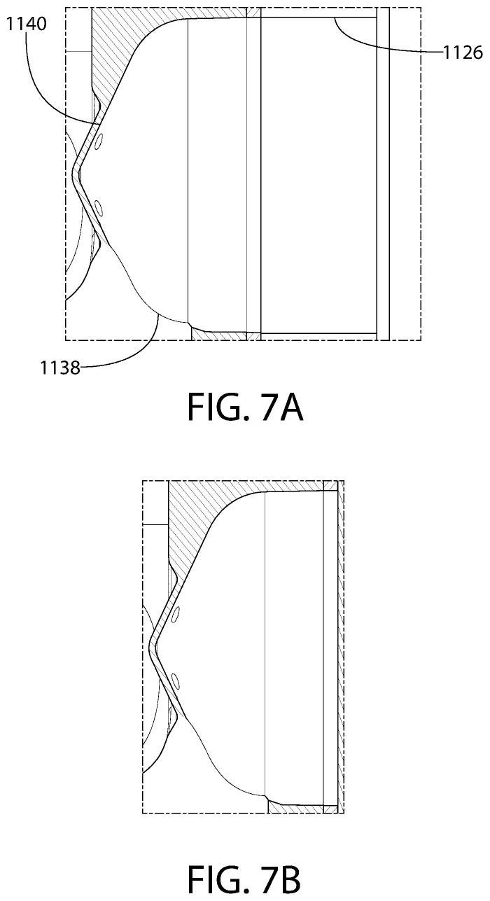

According to one embodiment, the internal height of the dosing chamber may be adjusted by lengthening the lower sidewall 1126, as shown in FIG. 7B. For example, the internal height of the dosing chamber may be between about 8 mm and about 12 mm, or between about 9 mm and about 11 mm. According to an embodiment shown in FIG. 7A, the internal height is between about 4 mm and about 6 mm, or between about 5 mm and about 6 mm, when the dosing chamber has a resonant frequency that is approximately the same as that of the transducer (between about 37 kHz and about 42 kHz). According to an alternative embodiment shown in FIG. 7B, when the transducer is activated at the same frequency of 37-42 kHz, the internal height of the dosing chamber is about twice the internal height shown in FIG. 7A, or about 1.7-2.3 times the internal height, or about 1.7-2.1 times the internal height e.g., between about 8 mm and about 12 mm, or between about 9 mm and about 11 mm. For example, it was found that a dosing chamber with an internal height of about 5.5 mm (X) had approximately the same resonant frequency as a dosing chamber with an internal height between about 9.9 mm (about 1.8X) and about 10.5 mm (about 1.9X), due to similar locations of anti-nodes at the opening(s), as evidenced by similar performance in synthetic jetting and dose delivery.

According to one embodiment, the synthetic jetting includes a maximum velocity when the transducer is activated for an unlimited amount of time. In some embodiments, the maximum velocity may be achieved in a relatively short amount of time of operating the transducer. In one embodiment, the maximum velocity is achieved when the transducer is activated for, e.g., from about 100 ms to about 1000 ms, or from about 100 ms to about 800 ms, or from about 100 to about 500 milliseconds.

According to one embodiment, the dosing chamber includes a vertical sidewall, wherein the vertical sidewall transitions to a shoulder, the shoulder being concave relative to a dosing chamber interior. The shoulder preferably transitions to a slope extending away from the sidewall and toward a center of the dosing chamber. Stated another way, the dosing chamber 1122 preferably includes a first portion 1128 having a lower sidewall 1126 (e.g., vertical sidewall), a second portion 1130 having an intermediate sidewall 1138 (e.g., comprising the shoulder), and a third portion 1132 having an upper sidewall 1140 (e.g., a slope extending away from the sidewall, radially disposed about axis 1124 and converging at a point 1136 to form a conical section). In one embodiment, the lower sidewall 1126 defines a cylindrical portion and the upper sidewall 1140 defines a conical portion. See, e.g., FIG. 6.

According to one embodiment, the slope transitions to an apex having a radius of curvature smaller than a radius of the shoulder. The dosing chamber further comprises one or more openings 1148 in the apex (e.g., between 1-10 openings, between 1-8 openings, between 1-6 openings, between 1-4 openings, between 2-10 openings, between 2-8 openings, between 2-6 openings, or between 2-4 openings). In an exemplary embodiment, the dosing chamber has 4 openings. As used herein, the term “apex” preferably refers to the conical portion of the dosing chamber defined by the upper sidewall 1140, which converges to a point 1136, i.e., the “apex” not only refers to the point 1136 but also to the conical portion defined by the upper sidewall that transitions to the point. The point of the apex is preferably rounded or pointed. The one or more openings positioned in the apex are preferably located closer to the point than to the shoulder.

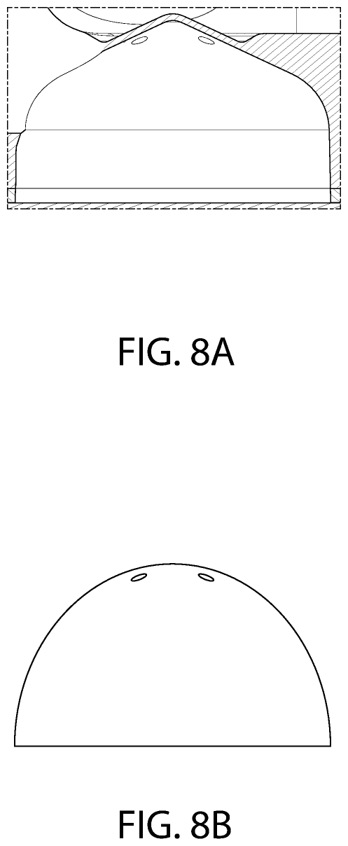

FIG. 8A provides an example of a dosing chamber with openings disposed in the apex 1136, contrary to FIG. 8B which shows a dosing chamber without an apex and openings instead disposed within a domed area that does not come to a point. It was found according to certain embodiments that synthetic jetting is improved when the openings (holes) of the dosing chamber are disposed within an apex rather than a flat top or a domed area that does not come to a point; for example, the maximum velocity is achieved when the transducer is activated for, e.g., from about 100 ms to about 1000 ms, or from about 100 ms to about 800 ms, or from about 100 to about 500 milliseconds. Without being bound by any theory, it is believed that the shape of the conical portion contributes to the achievement of suitable oscillating pressures (one or more anti-nodes) near the opening(s). Preferably, each of the plurality of openings has a centerpoint spaced equidistantly on a circle, the circle having its centerpoint on an axis defined by the apex.

The apex preferably has an apex wall thickness that is less than the wall thickness of the remainder of the dosing chamber, i.e., the conical portion of the dosing chamber comprising the opening(s) has a wall thickness that is the thickness of the vertical sidewall (also referred to as the lower sidewall), or less than the wall thickness of the remainder of the dosing chamber (e.g., the lower sidewall and intermediate sidewall comprising the shoulder). The aspect ratio of each opening, i.e., the length to cross-section or diameter of the passageway preferably is at least 0.5 and preferably is greater than or equal to about one, helps ensure that the mass of gas that moves back and forth in the passageway is created as discrete, well-formed slugs of air. It was found that the mechanical and acoustic energy of the transducer is more efficiently transferred to the dosing chamber when the walls of the dosing chamber, other than the apex, are not too thin, and have a thickness that is greater than the thickness of the portion of the apex where the opening(s) are disposed, in order to better retain vibratory energy.

According to one embodiment, the apex wall thickness is from about 0.002 inches (0.05 mm) to about 0.03 inches (0.8 mm), more preferably from about 0.004 inches (0.10 mm) to about 0.02 inches (0.5 mm), more preferably from about 0.004 inches (0.10 mm) to about 0.01 inches (0.25 mm), more preferably from about 0.006 inches (0.15 mm) to about 0.01 inches (0.25 mm). In one embodiment, the apex wall thickness is about 98% of the thickness of the largest wall thickness of the remainder of the dosing chamber. In one embodiment, the apex wall thickness is about 95% of the thickness of the largest wall thickness of the remainder of the dosing chamber. In one embodiment, the apex wall thickness is about 90% of the thickness of the largest wall thickness of the remainder of the dosing chamber. In one embodiment, the apex wall thickness is about 85% of the thickness of the largest wall thickness of the remainder of the dosing chamber. In one embodiment, the apex wall thickness is about 80% of the thickness of the largest wall thickness of the remainder of the dosing chamber. In one embodiment, the apex wall thickness is about 75% of the thickness of the largest wall thickness of the remainder of the dosing chamber. In one embodiment, the apex wall thickness is about 70% of the thickness of the largest wall thickness of the remainder of the dosing chamber. In one embodiment, the apex wall thickness is about 65% of the thickness of the largest wall thickness of the remainder of the dosing chamber. In one embodiment, the apex wall thickness is about 60% of the thickness of the largest wall thickness of the remainder of the dosing chamber. In one embodiment, the apex wall thickness is less than about 50% of the thickness of the largest wall thickness of the remainder of the dosing chamber. In one embodiment, the thickness of the remainder of the dosing chamber is substantially uniform.

According to one embodiment, the opening(s) fluidly connect the dosing chamber to the exit channel, wherein the aerosolized medicament is delivered from the dosing chamber to a user through the exit channel in response to an activation of the transducer. According to one embodiment, each opening has a diameter of from about 0.005 inches (0.13 mm) to about 0.05 inches (1.3 mm), or from about 0.008 inches (0.2 mm) to about 0.04 inches (1.0 mm), more preferably from about 0.01 inches (0.25 mm) to about 0.05 inches (1.3 mm), or from about 0.01 inches (0.25 mm) to about 0.04 inches (1.0 mm), or from about 0.01 inches (0.25 mm) to about 0.03 inches (0.76 mm); for example, about 0.019 inches (0.48 mm)±0.012 inches (0.30 mm), preferably from about 0.015 (0.38 mm) inches to about 0.03 inches (0.76 mm).

Embodiments of the dosing chamber are described in more detail below with reference to the Figures.

In one embodiment, the dosing chamber 1122 includes a first portion 1128 having a lower sidewall 1126, a second portion 1130 having an intermediate sidewall 1138, and a third portion 1132 having an upper sidewall 1140. In one embodiment, the lower sidewall 1126 defines a cylindrical portion and the upper sidewall 1140 defines a conical portion. In one embodiment, the dosing chamber 1122 includes an axis of symmetry 1124 about which at least a portion of the lower sidewall 1126, intermediate sidewall 1138, and upper sidewall 1140 of dosing chamber 1122 are disposed (e.g., radially disposed). Dosing chamber 1122 may therefore include a circular cross-section in at least one plane. The lower sidewall 1126 may have a vertical portion that extends from an outer surface of the dosing chamber housing. The first portion 1128, second portion 1130 and third portion 1132 may be a monolithic element, or may comprise one or more separate elements that are coupled together to form the dosing chamber; for example, the lower sidewall 1126 or a portion thereof may be coupled an element comprising the intermediate sidewall 1138 and upper sidewall 1140 to form the dosing chamber. In one embodiment, the dosing chamber housing includes a crown 1135 which defines a lower portion of the lower sidewall 1126.

In one embodiment, the height of the dosing chamber 1122 comprises the combined height of the first portion 1128, second portion 1130, and third portion 1132. In one embodiment, the lower sidewall 1126 defines a first portion height that is from 10%-75% of the chamber height. In one embodiment, the first portion height is from 20%-70% of the chamber height. In one embodiment, the first portion height is from 30%-65% of the chamber height. In one embodiment, the first portion height is from about 40%-60% of the chamber height. In one embodiment, the first portion height is from about 50%-55% of the chamber height. In one embodiment, a ratio of a height of the dosing chamber 1122 to a diameter of the first portion 1128 is from about 0.5 to about 0.65. In one embodiment, a ratio of a height of the dosing chamber 1122 to a diameter of the first portion 1128 is from about 0.55 to about 0.6. In one embodiment, a ratio of a length of the dosing chamber to a diameter of the first portion 1128 is from about 0.4 to about 0.75. In one embodiment, a ratio of the volume of the first portion 1128 to the combined volume of the second portion 1130 and the third portion 1132 is about 0.8 to about 1.3.

One or more openings 1148 are configured to extend through the upper sidewall 1140 to provide fluid communication between the dosing chamber 1122 and the exit channel 1182. Preferably, at least the area of the apex surrounding the opening(s) satisfies the following parameters for synthetic jetting described in U.S. Pat. No. 7,318,434: 1) The aspect ratio of each opening, i.e., the length to cross-section or diameter of the passageway preferably is at least 0.5 and preferably is greater than or equal to about one. In some embodiments, this aspect ratio helps ensure that the mass of gas that moves back and forth in the passageway is created as discrete, well-formed slugs of air; and 2) The distance the gas moves back and forth through the passageway preferably is greater than about two times the cross-section or diameter of the passageway. This helps to ensure that dry-powder disaggregated by the vortex created has a chance to escape the vortex's presence before the gas moves back through the passageway.

In one embodiment, the portion of the upper sidewall 1140 comprising the apex 1136 has a tapered thickness. For example, the upper sidewall 1140 could have a first thickness for a portion between the opening(s) and the intermediate sidewall and a second thickness (different from the first thickness) for the portion at or near the tip of the apex 1136. In one embodiment, the first thickness is greater than the second thickness. In one embodiment, the first thickness is less than the second thickness. In one embodiment, the upper sidewall 1140 may be stepped or abruptly change between the first thickness and the second thickness. In one embodiment, the upper sidewall 1140 gradually transitions between the first thickness and the second thickness. In one embodiment, the apex 1136 has a radius of curvature at the peak of the apex which is smaller than a radius of the intermediate sidewall 1138.

One or more openings 1148 are configured to extend through the upper sidewall 1140 to provide fluid communication between the dosing chamber 1122 and the exit channel 1182. In one embodiment, the openings 1148 each have a centerpoint equidistantly spaced on a circle (not shown) having its center on the axis 1124 of the chamber 1122 and a radius of about 0.5 mm to about 1.0 mm. In one embodiment, the chamber 1122 includes a single opening 1148. In one embodiment, the dosing chamber 1122 includes four openings 1148. In one embodiment, the openings 1148 are asymmetrically positioned about the axis 1124. In one embodiment, one of the openings 1148 is positioned on the axis 1124. In one embodiment, the opening 1148 has a diameter of about 0.019 inches (0.48 mm)±0.012 inches (0.30 mm), preferably from about 0.015 (0.38 mm) inches to about 0.03 inches (0.76 mm). In one embodiment, each of the openings 1148 have an opening sidewall disposed about its own opening axis of symmetry. In one embodiment, at least one of the openings 1148 has an opening axis of symmetry which is transverse to the axis 1124 of the dosing chamber 1122. In one embodiment, the opening axis of at least one of the openings 1148 is perpendicular to the upper sidewall 1140. In one embodiment, the dosing chamber 1122 includes more than one opening 1148, each having an axis which may all be parallel to each other, one not parallel to the others, each perpendicular to the surface of the upper sidewall 1140, and/or one parallel to the chamber axis 1124. In one embodiment, the diameter of the openings 1148 may be influenced by the number of openings in the apex 1136. For example, a chamber 1122 having two openings 1148 may have greater opening diameters than a chamber having four openings such that the dosing chamber has a consistent total opening surface area regardless of the number of openings. The openings 1148 are configured to have any desired shape (e.g., circular, elliptical, rectangular, etc.) provided that they allow an aerosolized pharmaceutical to pass therethrough. In one embodiment, the openings 1148 have a size selected to ensure that the pharmaceutical is of a size to permit it to pass to the lungs of a user.

The dosing chamber may be manufactured from a single material or different portions may be made from different materials. For example, the lower sidewall 1126, intermediate sidewall 1138, and upper sidewall 1140 of the dosing chamber 1122 may comprise a first material and the upper surface of the housing may comprise a second material.

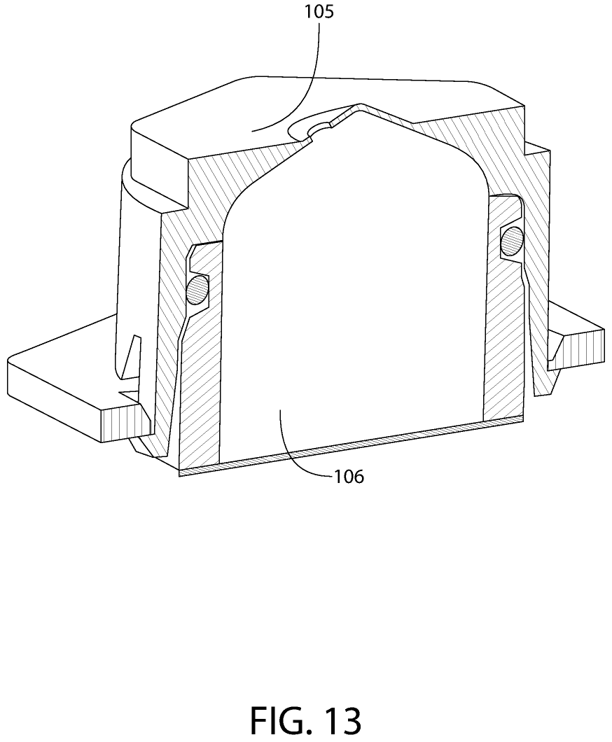

In a preferred embodiment of the single-dose cartridge inhaler, the dosing chamber is manufactured from at least two different portions that are coupled together, namely, a bottom portion 106 and a top portion 105, with the top portion comprising the one or more openings. Preferably, a dose of dry powder medicament is dispensed into the bottom portion 106, and subsequently the top portion 105 is attached to the bottom portion via welding, adhesive, fasteners or the like. Together, the interior of the top portion and the interior of the bottom portion preferably form the whole interior of the dosing chamber, as illustrated in FIG. 13.

According to an embodiment, the opening(s) in the dosing chamber are sealed by a sealing mechanism so that the dose of medicament inside the dosing chamber, or a portion thereof, is prevented from leaking out of the opening(s) prior to the administration of a dose. The opening(s) in the top portion of the dosing chamber are preferably sealed by the sealing mechanism prior to assembling the top portion onto the bottom portion during the manufacturing process. Alternatively, the opening(s) in the top portion of the dosing chamber may be sealed by the sealing mechanism after assembling the top portion onto the bottom portion. According to one embodiment, the sealing mechanism comprises a silicone seal 108 that overlays or covers the opening(s) in the dosing chamber 1122. According to an embodiment, the sealing mechanism 108 is removed from the opening(s) prior to the administration of a dose of medicament (e.g., moved away from the opening(s) so that medicament is not prevented from exiting the dosing chamber). For example, the sealing mechanism may be moved away from the dosing chamber holes upon attachment of the cartridge to the base. In one embodiment, a seal is attached to a brace, and the brace is pushed away from the openings as the cartridge is being attached to the base, because a protrusion on the brace comes into contact with a protrusion in the base, which forces the brace and seal away from the opening(s). Alternatively, a subject may remove the sealing mechanism manually, for example, by pulling a tab that is affixed to the sealing mechanism.

According to preferred embodiments, a membrane is adhered to the dosing chamber and couples the chamber to the vibrating element. As used herein, a membrane is preferably a sheet of material disposed between the face of the transducer and the inside of the dosing chamber (e.g., as a partition), wherein the sheet of material is preferably pliable. The membrane preferably meets the following criteria: biocompatible; compliant material that effectively converts vibration to appropriate levels of acoustic activity; robustness to damage; reliable adhesion to the dosing chamber material; and having an appropriate coefficient of thermal expansion. Preferably, the membrane is capable of retaining tension and adhesion under expected environmental conditions throughout the inhaler's intended life, and remains smooth and flat while providing effective vibratory transfer to the dosing chamber acoustic resonance. Overall, the material and tension of the membrane should optimize energy transfer from the transducer to the dosing chamber, so that a fast onset of synthetic jetting and delivered dose uniformity can be achieved.

According to one embodiment, the inhaler comprises: a dosing chamber configured to receive medicament; a transducer confronting the dosing chamber, the transducer being configured to aerosolize the medicament when the transducer is activated; and a membrane between the dosing chamber and the transducer, the membrane being stretched across a dosing chamber opening and affixed to the dosing chamber; wherein the device produces a synthetic jet to deliver the aerosolized medicament to a user when the transducer is activated.

According to another embodiment, the medicament delivery device comprises a dosing chamber comprising an interior that is configured to contain dry powder medicament; a transducer confronting the dosing chamber, wherein the dosing chamber and the transducer are acoustically resonant such that the dosing chamber is configured to resonate in response to an activation of the transducer; and a membrane between the dosing chamber and the transducer, the membrane being stretched across a dosing chamber opening (and preferably affixed to the dosing chamber), the membrane being comprised of a material having a thickness. According to this embodiment, the membrane material and thickness affect the deaggregation and/or delivery of powder. For example, one or more of the speed of onset of synthetic jetting, maximum synthetic jetting, delivered dose per burst, total delivered dose, and aerodynamic particle size distribution may be affected by a change in the membrane material or thickness. Preferably, the membrane material and its thickness are selected such that the combined acoustic resonance of the transducer, dosing chamber and membrane is sufficient to cause aerosolization and delivery of the dry powder medicament having an MMAD within the preferred ranges described herein, e.g., about 6 μm or less, preferably with a fine particle fraction within ranges described herein, e.g., at least 30%. Maximum synthetic jetting is preferably achieved within the preferred ranges of time described herein, e.g., within about 500 ms or less from the start of a transducer activation.

According to one embodiment, the membrane has a tensile strength (MD or machine direction) of at least 30 MPa, more preferably at least 40 MPa, or at least 50 MPa, or at least 60 MPa, or at least 70 MPa, or at least 80 MPa, or at least 90 MPa, or at least 100 MPa, or at least 120 MPa, or at least 150 MPa, or at least 200 MPa; for example from about 30 MPa to about 200 MPa, or from about 40 MPa to about 200 MPa. According to one embodiment, the membrane has a tensile modulus of 7.0 GPa or less, or 6.0 GPa or less, or 5.0 GPa or less; for example, from about 1.0 GPa to about 7.0 GPa, or from about 1.0 GPa to about 6.0 GPa. According to one embodiment, the membrane has a tensile elongation at yield (MD or machine direction) of at least 50%, or at least 75%, or at least 100%; for example, from about 50% to about 300% elongation, or from about 75% to about 300% elongation at yield, or from about 100% to about 300% elongation at yield. According to one embodiment, the membrane has a coefficient of thermal expansion (CTE) of less than 120 ppm/° C., or less than 100 ppm/° C., or less than 90 ppm/° C., or less than 80 ppm/° C., or less than 70 ppm/° C.; for example, from about 10 ppm/° C. to about 100 ppm/° C., or from a bout 10 ppm/° C. to about 90 ppm/° C., or from about 10 ppm/° C. to about 80 ppm/° C., or from about 10 ppm/° C. to about 70 ppm/° C. According to one embodiment, the membrane has a Tg (glass transition temperature) of at least 60° C., or at least 70° C., or at least 80° C., or at least 90° C., or at least 100° C.; from example, from about 50° C. to about 250° C., or from about 60° C. to about 250° C., or from about 60° C. to about 200° C., or from about 60° C. to about 175° C.

According to one embodiment, the membrane has one or more of the following characteristics: a tensile strength (MD) of at least 30 MPa, a tensile modulus of 7.0 GPa or less, a tensile elongation (MD) of at least 50%, a CTE of less than 100 ppm/° C., and a Tg of at least 60° C. Non-limiting examples of such materials include polyethelyne terephthalate (PET) (e.g., Mylar® 813), polyether ether ketone (PEEK) (e.g., APTIV® 2000-050), polycarbonate (e.g., LEXAN® SD8B14), polysulfone (e.g., Udel®), polyetherimide (e.g., ULTEM®), polyvinylidene fluoride (e.g., KYNAR®), and polyvinyl chloride.

According to another embodiment, the membrane has one or more of the following characteristics: a tensile strength (MD) of at least 40 MPa, a tensile modulus of 6.0 GPa or less, a tensile elongation (MD) of at least 75%, a CTE of less than 100 ppm/° C., and a Tg of at least 70° C. Preferably, the membrane is a material that is heat sealable to the dosing chamber. In one embodiment, the membrane is manufactured from one of polyethelyne terephthalate (PET) (e.g., Mylar® 813), polyether ether ketone (PEEK) (e.g., APTIV® 2000-050), polycarbonate (e.g., LEXAN® SD8B14), polysulfone (e.g., Udel®), polyetherimide (e.g., ULTEM®), polyvinylidene fluoride (e.g., KYNAR®), polyvinyl chloride, or similar material with one or more of the properties described herein (e.g., tensile strength, tensile modulus, tensile elongation, CTE, Tg, etc.).

According to one embodiment, the membrane is under a tensile force of about 0.15 N/mm to about 1.0 N/mm, or 0.2 N/mm to about 1.0 N/mm, or from about 0.2 N/mm to about 0.8 N/mm, or from about 0.2 N/mm to about 6 N/mm (as measured when assembled with the dosing chamber). According to one embodiment, the membrane has a thickness of about 30 um to about 150 um, or about 40 um to about 100 um, or about 40 um to about 70 um, or about 40 um to about 60 um, or about 50 um to about 80 um. According to one embodiment, the membrane material is selected from at least one of PET, polycarbonate and PEEK. According to another embodiment, the membrane material is selected from at least one of PET and polycarbonate. According to one embodiment, the membrane has a membrane thickness which is about 0.38% to about 0.43% of a chamber height. Embodiments of the membrane are described in more detail below with reference to the Figures.

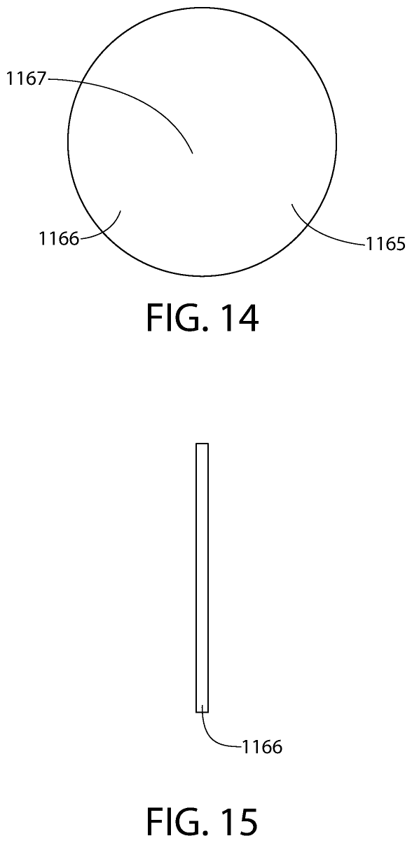

In one embodiment, the membrane 1166 is coupled to the dosing chamber housing and covers the opening to the dosing chamber 1122 such that the membrane is vibrated when the transducer 150 is activated. Turning now to FIGS. 14-15, a membrane 1166 (or film) is shown. The membrane 1166 is configured to be coupled to the outer surface of the housing 1102 such that the membrane 1166 covers the open end 1170 of the dosing chamber 1122. The membrane 1166 is configured to be coupled to the dosing chamber housing via adhesive, welding, anchors, etc. In one embodiment, the membrane 1166 is shaped similarly to the open end 1170 of the dosing chamber 1122 such that the membrane completely covers the open end. In one embodiment, the membrane 1166 has a uniform thickness. In one embodiment, some portions of the membrane 1166 are thicker or thinner than other portions.

In one embodiment, the membrane 1166 is manufactured from a material that allows it to be stretched across the dosing chamber opening to promote efficient energy coupling between the transducer and membrane when the transducer vibrates. In one embodiment, the membrane 1166 is manufactured from one of polyethelyne terephthalate (PET) (e.g., Mylar® 813), polyether ether ketone (PEEK) (e.g., APTIV® 2000-050), polycarbonate (e.g., LEXAN® SD8B14), polysulfone (e.g., Udel®), polyetherimide (e.g., ULTEM®), polyvinylidene fluoride (e.g., KYNAR®), polyvinyl chloride, or similar material provided that the membrane can be stretched across at least a portion of the open end 1170. In one embodiment, the membrane 1166 is under a tensile load when it is initially stretched across the open end 1170. In one embodiment, the tension is about 0.17 to about 1.09 N/mm. In one embodiment, the tension is about 0.17 N/mm to about 1.09 N/mm when the inhaler 100 is not in use. The tension value may be selected based, at least in part, by the material or thickness of the membrane 1166. For example, tension may be selected based on the membrane material and the resultant resonant frequency of a membrane having the selected material and tension such that the resonant frequency of the membrane approximates the resonant frequency of the transducer 150, as explained in greater detail below. In one embodiment, the membrane 1166 is opaque. In one embodiment, the membrane 1166 is translucent or semi-translucent. In one embodiment, the membrane 1166 comprises more than one layer of the same or different materials.

The membrane 1166 is preferably configured to be coupled to the crown 1135. The peel strength between the membrane 1166 and the crown 1135 may be selected to ensure that the membrane 1166 does not disengage from the crown 1135 after a selected number of uses when the membrane is vibrated. The peel strength may also be selected to reduce the likelihood of air entering or escaping the dosing chamber 1122 between the membrane 1166 and the crown 1135. The peel strength in one embodiment is configured to be about 75 g to about 250 g. In one embodiment, the portion of the membrane coupled to the crown is treated (e.g., chemical etching, physical scoring) prior to coupling to improve the bond between the elements. In one embodiment, the outer region 1165 of the membrane 1166 is thicker than the inner region 1167 and the outer region 1165 is treated (e.g, chemical etching, physical scoring) prior to coupling the membrane 1166 to the crown 1135. In one embodiment, the membrane 1166 comprises a sheet which is secured to the crown 1135 and trimmed in place such that the membrane has the same outer diameter as the crown 1135. In one embodiment, the membrane 1166 is wrapped around the edges and secured to the sides of the crown 1135. In one embodiment, the membrane 1166 includes a membrane effective area. In an embodiment, the membrane effective area is the portion of the membrane 1166 inside of the inner face of the crown 1135 above the open end 1170 of the dosing chamber 1122 such that the membrane effective area can move (or vibrate) without contacting the crown 1135. In one embodiment, the membrane thickness is about 0.1% to about 1%, or about 0.1% to about 0.8%, or about 0.1% to about 0.6%, or about 0.2% to about 1%, or about 0.2% to about 0.8%, or about 0.2% to about 0.6%, or about 0.38% to about 0.43% of the chamber height.

According to one embodiment, the membrane material is a PET material having a thickness from about 10 μm to about 40 μm; or a polycarbonate material having a thickness from about 20 μm to about 60 μm, wherein the material is heat sealed to the dosing chamber with an adhesive (e.g., Loctite® 4310). For example, it was found that a PET material (Mylar® 813) having a nominal thickness of about 23±10 μm or a polycarbonate material (LEXAN® Sabic SD8B14) having a nominal thickness of about 50±15 μm enabled optimal synthetic jetting (e.g., maximum synthetic jetting of at least 0.5 V in response to an activation of the transducer) and dose delivery.

The inhaler of the present invention comprises an air flow conduit (used interchangeably with the term “flow channel”), which preferably extends from an air inlet (an opening through which air is drawn into the air flow conduit when a user inhales through the device) to an outlet (an opening through which air entrained with dry powder medicament exits the inhaler's mouthpiece). The size and shape of the air flow conduit are designed to achieve the desired flow resistance (e.g., suitable for patients with COPD or cystic fibrosis), accommodate the position of the aerosol engine within the inhaler, and provide a flow path from the dosing chamber to the outlet that does not obstruct the flow of dry powder. The flow resistance provided by the air flow conduit is preferably low enough to be comfortable for patients that have difficulty inhaling (e.g., COPD patients, cystic fibrosis patients, etc.) but high enough to be detectable by the flow sensor.

According to an exemplary embodiment, the air flow conduit provides a flow resistance from about 0.040 cmH₂O^{0.5}/LPM to about 0.12 cmH₂O^{0.5}/LPM, or from about 0.040 cmH₂O^{0.5}/LPM to about 0.1 cmH₂O^{0.5}/LPM, or from about 0.040 cmH₂O^{0.5}/LPM to about 0.090 cmH₂O^{0.5}/LPM, or from about 0.050 cmH₂O^{0.5}/LPM to about 0.12 cmH₂O^{0.5}/LPM, or from about 0.050 cmH₂O^{0.5}/LPM to about 0.1 cmH₂O^{0.5}/LPM, or from about 0.050 cmH₂O^{0.5}/LPM to about 0.090 cmH₂O^{0.5}/LPM, or from about 0.040 cmH₂O^{0.5}/LPM to about 0.085 cmH₂O^{0.5}/LPM, or from about 0.050 cmH₂O^{0.5}/LPM to about 0.085 cmH₂O^{0.5}/LPM, or from about 0.060 cmH₂O^{0.5}/LPM to about 0.085 cmH₂O^{0.5}/LPM, or from about 0.070 cmH₂O^{0.5}/LPM to about 0.085 cmH₂O^{0.5}/LPM, or from about 0.070 cmH₂O^{0.5}/LPM to about 0.09 cmH₂O^{0.5}/LPM, or from about 0.070 cmH₂O^{0.5}/LPM to about 0.095 cmH₂O^{0.5}/LPM, or from about 0.070 cmH₂O^{0.5}/LPM to about 0.1 cmH₂O^{0.5}/LPM, or from about 0.070 cmH₂O^{0.5}/LPM to about 0.12 cmH₂O^{0.5}/LPM at a flow rate of about 30 liters per minute (LPM).

According to an embodiment, the air flow conduit has a constricted section 1400 with a cross-sectional area that is significantly less than the cross-sectional area(s) of the remainder of the air flow conduit, and which provides flow resistance. The placement of the constricted section within the air flow conduit typically affects the delivery of powder from the dosing chamber. In certain embodiments described in U.S. Pat. NO. 15/729,526, and according to certain embodiments of the present invention, the constricted section is upstream from the area of the air flow conduit into which dry powder medicament is expelled from the opening(s) of the dosing chamber, e.g., the constricted section is not in the area of the air flow conduit where the dry powder medicament is expelled from the dosing chamber, in order to prevent a pressure drop over the dosing chamber. Such an embodiment is illustrated in FIG. 16, in which the general direction of air flow is illustrated by arrow 1403, wherein the air flows in a direction from upstream 1401 (e.g., inlet) to downstream 1402 (e.g., outlet). For example, the constricted section 1400 may be formed by at least one ledge 1194 or at least one protrusion that extends into the air flow conduit and creates a narrow cross-sectional area (e.g., there may be one ledge as shown in FIG. 16, or alternatively more than one ledge extending into the air flow conduit to create the constricted section 1400). In one embodiment, the constricted section 1400 is located in the upstream area 1401 of the air flow conduit, i.e., upstream from the area of the air flow conduit into which dry powder medicament is expelled from the opening(s) of the dosing chamber. In an embodiment, the constricted section 1400 is also located upstream from the flow sensor or from an aperture 1190 that provides fluid communication between the air flow conduit and the flow sensor. For example, the constricted section 1400 may be disposed at the air inlet 1191 (see e.g. FIG. 16), or near the air inlet 1191 (e.g., downstream from the air inlet and upstream from the flow sensor).

In accordance with alternative embodiments of the single-use cartridge inhaler described herein, the constricted section 1400 is disposed over the dosing chamber opening(s), or adjacent to the dosing chamber opening(s), in order to cause a pressure drop over the opening(s) which aids in drawing dry powder medicament out of the dosing chamber. This is particularly preferable when the dose of medicament is large, e.g., when the dose is at least 10 mg, at least 20 mg, at least 30 mg, at least 40 mg, or at least 50 mg, although it may be employed for smaller doses. The constricted section aids in lifting powder out of the dosing chamber into the air flow conduit when a subject inhales through the air flow conduit, because a pressure drop occurs at the constricted section. In certain embodiments, an air flow conduit comprising a constricted area over the dosing chamber opening(s) or adjacent the opening(s) may be referred to as a high-velocity flow channel. In accordance with this embodiment, it has been found that the inhaler is capable of delivering a dose of dry powder medicament by a combination of both active delivery and passive delivery. Active delivery is achieved as a result of activation of the transducer and transfer of mechanical and/or acoustic energy to the dosing chamber, as described herein. Passive delivery is achieved as a result of the constricted area being disposed over the dosing chamber opening(s) or adjacent the opening(s), which causes powder to be lifted out of the dosing chamber regardless of whether the transducer is activated. It has been found that an amount of powder can be delivered by active delivery alone (i.e., an “active amount,” regardless of where the constricted section is located), and an amount of powder can be delivered by passive delivery alone (i.e., a “passive amount” without transducer activation and via inhalation only), wherein the amount of powder delivered by both active delivery and passive delivery together, when the transducer is activated, is equal to, or greater than, the active amount and passive amount combined.

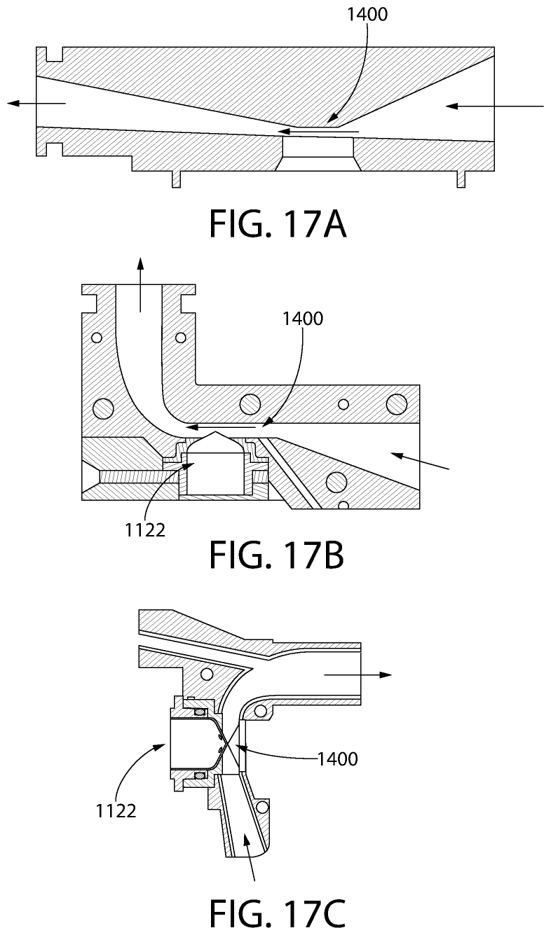

In an embodiment shown in FIGS. 17A-17C, the constricted section 1400 is located in the area of the air flow conduit where the dry powder medicament is expelled (e.g., over the dosing chamber opening(s) or adjacent the opening(s)), so that a pressure drop is induced over the dosing chamber by the reduced cross-sectional area (e.g., a high-velocity flow channel). The arrows in FIGS. 17A-17C illustrate the direction of air flow when a subject inhales through the device.

The medicament delivery device of the present invention comprises a mounting system for the vibrating element (e.g., transducer). Several challenges were faced during the development of a suitable mounting system that would couple the vibrating element to the inhaler housing and apply sufficient pressure to the vibrating element so that its mechanical and acoustic energy would transfer to the dosing chamber, but without applying so much pressure that the vibratory energy would be dampened. A piezoelectric transducer, for example, should be mounted to the inhaler in a manner that does not interfere with vibratory output, that is compatible with high volume manufacturing methods, and that can be retained in the inhaler when the removable cartridge is not attached. The mounting system of the present invention is designed for minimal contact with the transducer housing to prevent attenuation of vibration. According to a preferred embodiment, a spiral wave spring provides force with a low profile and reasonably low spring rate to save space and allow reasonable robustness, e.g., a coil spring would typically require a greater length in order to provide the same amount of force. The transducer mounting system enables the transducer to be held in place when a cartridge is not attached while maintaining a sufficient pre-load force throughout the use life of the inhaler.

According to one embodiment, the inhaler comprises: a housing; a dosing chamber configured to receive a medicament; a transducer confronting the chamber, the transducer being configured to aerosolize the pharmaceutical when the transducer is activated; a holder configured to secure the transducer to the housing; and a biasing element between the holder and the housing.

According to another embodiment, the medicament delivery device comprises a housing; a dosing chamber configured to contain dry powder medicament; a transducer confronting the dosing chamber, wherein the dosing chamber and the transducer are preferably acoustically resonant such that the dosing chamber is configured to resonate in response to an activation of the transducer; and a transducer mounting assembly. The transducer mounting assembly preferably comprises (i) a holder configured to secure the transducer to the housing and (ii) a biasing element disposed between the holder and the housing. The biasing element presses the transducer against the dosing chamber with sufficient force to cause vibratory energy to be transferred from the transducer to the dosing chamber upon an activation of the transducer so that the dry powder medicament can be aerosolized and delivered from the dosing chamber via synthetic jetting (e.g., the dosing chamber resonates with acoustic energy, as evidenced by synthetic jetting, and mechanical vibrations). The holder provides additional surface area for the biasing element to interact with the transducer; for example, if the biasing element is a spring, the spring may not have enough surface area where it touches the transducer to sufficiently press the transducer against the dosing chamber. Preferably, less than half the outer surface area of the transducer is in contact with the holder. Embodiments of a transducer mounting system suitable for use with the inhaler of the present invention are described in U.S. Pat. No. 15/729,526.

As discussed herein, the inhaler of the present invention preferably employs synthetic jetting to aerosolize the drug powder. There exist the needs to 1) shorten the onset time for establishing the synthetic jet and delivering the drug in response to a patient's breath actuation (dose trigger); 2) conserve energy; 3) more effectively deagglomerate the drug formulation to ensure a consistent particle size distribution of the delivered dose; and 4) ensure consistent dosing and particle size distribution throughout the life of the device. During development of the present invention, extensive studies were conducted to couple the energy of the vibratory element (transducer) to the dosing chamber so that these objectives could be achieved. By providing an air column that extends between the transducer and the membrane, wherein at least a portion of the air column is defined by a separation means (e.g., spacer 1286), it was discovered that the air column increases power draw by allowing for higher displacement of the transducer face and membrane without contact between the two. It was also discovered that, in preferred embodiments, the air column shortens the onset time for establishing the synthetic jet and delivering the drug in response to a patient's breath actuation. This was found to be a particular advantage for those patients that perform short inhalations through the device during tidal breathing. An exemplary spacer that may be used in one embodiment is described in WO 2016/007356, which is incorporated by reference herein.

A spacer is not required for the aerosol engine to achieve sufficient synthetic jetting, dose delivery and aerodynamic particle size distribution, but it is an optional feature that may increase the overall robustness of the aerosol engine. For example, the inhaler's aerosol engine may still achieve maximum synthetic jetting within less than 1000 ms, less than 500 ms, or less than 100 ms without a spacer if the acoustic resonance of the system as a whole allows for sufficient energy transfer from the transducer to the dosing chamber. According to certain embodiments, the spacer shortens the amount of time to maximum synthetic jetting and/or increases the maximum synthetic jetting; for example, the amount of time to maximum synthetic jetting may be reduced by at least 10 ms, or at least 20 ms, or at least 30 ms, or at least 40 ms, or at least 50 ms when a spacer is employed.

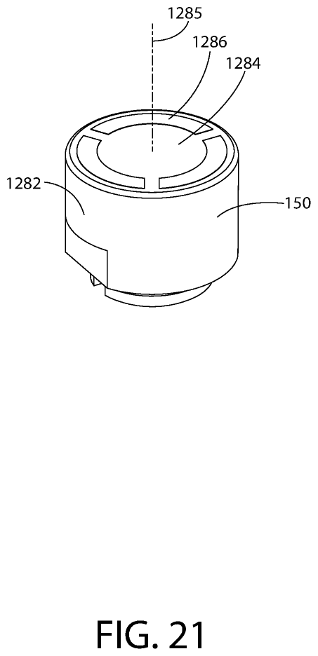

According to an embodiment, the inhaler of the present invention comprises a dosing chamber configured to receive medicament; a transducer confronting the dosing chamber, the transducer being configured to aerosolize the medicament when the transducer is activated; a membrane disposed between the dosing chamber and the transducer, the membrane being affixed to the dosing chamber; and an air column extending between the transducer and the membrane, wherein at least a portion of the air column is defined by a separation means (e.g., a spacer), wherein the inhaler produces synthetic jetting to deliver the aerosolized medicament to a user when the transducer is activated. Embodiments of the spacer are described in more detail below with reference to the Figures. Preferably, the spacer is in contact with both the face of the transducer and the membrane. As described below, the spacer height (e.g., measured between the face of the transducer and the membrane) is preferably about 10 μm to about 100 μm. Synthetic jetting may be measured in accordance with known methods, such as the method described in Example 1. In some embodiments, the inhaler 100 includes a spacer 1286 between the transducer 150 and the membrane 1166 to enhance the transfer of acoustic vibration and physical vibration between the transducer 150 and the membrane 1166. In some embodiments, the presence of air between the transducer 150 and membrane 1166 enhances the vibrational energy transfer between the two, thus, in some embodiments, the inhaler 100 does not include a spacer but a gap is provided between the transducer and membrane. In one embodiment the transducer 150 comprises a piezoelectric transducer. Piezoelectric transducers are well-known and readily available to those skilled in the art. According to one embodiment, the piezoelectric transducer resonates at approximately 37 to approximately 43 kHz, or approximately 38 to approximately 41 kHz. An embodiment of a transducer comprising a spacer is illustrated in FIG. 21. In one embodiment, the transducer 150 includes a cylindrical body 1282 and a transducer face 1284 having an axis of symmetry 1285. In one embodiment, a spacer 1286 is positioned on the transducer face 1284. In one embodiment, the spacer 1286 and the transducer face 1284 are a monolithic element. In one embodiment, the spacer 1286 is a dielectric ink (e.g., Acheson ML25240 UV Cure Dielectric Ink, electrically non-conductive ink) and is screen printed onto the transducer face 1284.

In a preferred embodiment, the vibration of the transducer 150 is configured to transfer the vibratory energy through physical vibration of the housing 1102 as well as through acoustic vibration as previously described. The transfer of vibrational energy through the inhaler 100 may be made more efficient by matching resonant frequency across the various components of the system. Vibrating an element at its resonant frequency will amplify the vibration of the element. Some vibration is cancelled out when an element is vibrated at a frequency other than its resonant frequency. A system with elements that each have the same (or common) resonant frequency may achieve synthetic jetting faster when the system is driven at the common resonant frequency than a system with elements having mismatched resonant frequencies. In some embodiments, the inhaler 100 includes elements (e.g., transducer, dosing chamber, membrane and air column) which have a common resonant frequency to efficiently transfer vibrational energy throughout the system. The transducer 150 may be characterized by an acoustic resonant frequency (or resonant frequency). In one embodiment, the features (e.g., dimensions, materials, orientation) of each of the spacer 1286, the membrane 1166, and the dosing chamber 1122 are adjusted such that the resonant frequency of each component, as well as the resonant frequency of the system comprised of these components, is matched or is closely related to the resonant frequency of the transducer 150. For example, without being limited by any particular theory, changing the material used for any of the components may affect the resonant frequency of each component and/or the overall system. However, this does not mean that a common resonant frequency for the system cannot be achieved simply because the materials comprising the elements are substituted. Instead, other elements or features of the system can be changed to re-coordinate the resonant frequency of the system. For example, changing the height or width or wall thickness of the dosing chamber also affects the resonant frequency of the dosing chamber 1122 and the system. Thus, the material used to manufacture the housing 1102 containing the dosing chamber 1122 could be changed, and the dimensions of the dosing chamber also changed to maintain the resonant frequency of the dosing chamber and the system. Any element of the system may be changed and one or more of the remaining elements of the system may also be changed to maintain a common resonant frequency of each element and across the system. Individual elements, segments of the system, and/or the system as a whole, may be configured to have more than one resonant frequency or harmonic which may be a multiple of the first resonant frequency.

In one embodiment, the desired resonant frequency is selected by choosing a transducer 150, determining its resonant frequency, and then configuring a system which has a similar resonant frequency. In one embodiment, a dosing chamber is configured to fit within a desired inhaler, or a dosing chamber is manufactured from a certain material that will avoid negative interactions with a pharmaceutical is chosen and the rest of the components and system are configured to match the resonant frequency of the dosing chamber. In one embodiment, the resonant frequency of the system is determined when there is no pharmaceutical within the dosing chamber. In one embodiment, the resonant frequency of the system is determined when the dose of medicament is inside the dosing chamber. In one embodiment, a system having the same or a similar acoustic resonance reduces the onset time to establish synthetic jetting and reduce the battery power needed to deliver a pharmaceutical to a user through the inhaler.

Acoustic impedance is generally the relationship between the acoustic pressure applied to a system and the resulting particle velocity in the direction of that pressure at its point of application. Acoustic impedance is generally defined as Z₀=ρ₀·c₀ where Z₀ is acoustic impedance in units of Rayls (Pa·s/m); ρ₀ is density of the medium (kg/m3); and c₀ is the speed of sound through the medium (m/s). A system that has identical or a small variation in acoustic impedance across the elements of the system creates a more efficient energy transfer (or energy coupling) during operation of the system. The onset time for synthetic jetting is reduced in a system with greater acoustic impedance matching compared to a system with less acoustic impedance matching. The acoustic impedance may be thought of as the “stiffness” of each element. When the acoustic impedance is matched or is within a narrow range, the elements of the system (e.g., the air column, membrane, and the air within the chamber) can move in relative unison as the transducer vibrates, thus each vibration of the transducer may transfer more vibration energy to the air within the dosing chamber.

In accordance with particular embodiments of the present invention, it was discovered that dry powder tends to get “stuck” in low pressure nodes of the dosing chamber (those areas with little or no oscillating pressure), which causes the synthetic jetting and resulting delivered dose to decrease substantially. It was further discovered that the drive scheme could be changed in a way that addresses this problem; specifically, the resonant frequency of the transducer is periodically interrupted of “switched off” to a non-resonant frequency (or “hop frequency”), according to particular embodiments. Switching off the resonant frequency interrupts the levitation of the particles so that they do not remain stuck in the low pressure nodes. According to preferred embodiments, the inclusion of a hop frequency significantly improves the gravimetric clearance of powder out of a dose. For example, a drive scheme without a hop frequency may result in a gravimetric clearance of less than 50%, or less than 40% of powder from a dose, whereas a drive scheme including a hop frequency may result in a gravimetric clearance of greater than 60%, preferably greater than 70%, or greater than 80% or greater than 90% or greater than 95% of powder from a dose.

According to one embodiment, a method of driving a piezoelectric transducer in a medicament delivery device comprises: activating the transducer by providing an electric signal to the transducer for a period of time, wherein the electric signal provides a first frequency which causes the transducer to oscillate at its resonant frequency, and a second frequency that is different from the first frequency and does not cause the transducer to oscillate at its resonant frequency, wherein the electric signal alternates between the first frequency and the second frequency during said period of time. According to an additional embodiment, a medicament delivery device comprises a dosing chamber comprising an interior that is configured to contain dry powder medicament; a transducer confronting the dosing chamber, wherein the dosing chamber and the transducer are acoustically resonant such that the dosing chamber is configured to resonate in response to an activation of the transducer; and a controller configured to send an electric signal to the transducer that alternates between a first frequency and a second frequency during a transducer activation, wherein the first frequency causes the transducer to oscillate at its resonant frequency, and the second frequency is different from the first frequency and does not cause the transducer to oscillate at its resonant frequency (e.g., the device contains a program code capable of generating said electric signal).

Preferably, the signal alternates between the first frequency and the second frequency multiple times during a transducer activation. The second frequency may be referred to as a “hop frequency.” The use of a hop frequency preferably causes aerosolization and delivery of the dry powder medicament having an MMAD within the preferred ranges described herein, e.g., about 6 μm or less, preferably with a fine particle fraction within the preferred ranges described herein, e.g., at least 30%. Maximum synthetic jetting is preferably achieved within ranges of time described herein, e.g., within about 500 ms or less from the start of a transducer activation. According to an embodiment, maximum synthetic jetting and/or speed of onset of synthetic jetting is greater for a device that employs a hop frequency than a device that does not employ a hop frequency. Delivered dose per burst, total delivered dose, and aerodynamic particle size distribution may also be improved when a hop frequency is used.

Preferably, the first frequency is substantially equivalent to the resonant frequency of the piezoelectric transducer; and the second frequency is not substantially equivalent to the resonant frequency of the piezoelectric transducer. A frequency that is substantially equivalent to the resonant frequency of the piezoelectric transducer refers to a frequency that is equal to the resonant frequency of the piezoelectric transducer, or sufficiently close to the resonant frequency of the piezoelectric transducer that it causes the transducer to produce oscillations that are sufficient to generate synthetic jetting.

According to one example, the transducer's resonant frequency is between 37-42 kHz; during a dosing breath, the transducer is activated by an electric signal having a first frequency that is also between 37-42 kHz, and the first frequency is subsequently “interrupted” by a second frequency that is outside the range of 37-42 kHz, i.e., less than 37 kHz or greater than 42 kHz. During a dosing breath, the first frequency is provided for the majority of the transducer's “on-time” while the second frequency briefly interrupts the first frequency intermittently so that dry powder particles do not remain stuck in low pressure nodes inside the dosing chamber. A brief interruption by the second frequency (“hop frequency”) is still considered part of the on-time.

According to one embodiment, the method comprises activating the transducer for from about 50 ms to about 1000 ms upon each dosing breath; for example from about 50 ms to about 900 ms, or about 50 ms to about 800 ms, about 50 ms to about 700 ms, or about 50 ms to about 600 ms, or about 50 ms to about 500 ms, or about 50 ms to about 400 ms, or about 50 ms to about 300 ms, or about 50 ms to about 200 ms, or about 50 ms to about 100 ms, or about 100 ms to about 900 ms, or about 100 ms to about 800 ms, or about 100 ms to about 700 ms, or about 100 ms to about 600 ms, or about 100 ms to about 500 ms, or about 100 ms to about 400 ms, or about 100 ms to about 300 ms, or about 100 ms to about 200 ms upon each dosing breath. As described herein, an inhalation cycle preferably includes multiple dosing breaths.

According to one embodiment, the method comprises providing the first frequency for at least about 70%, or at least about 75%, or at least about 80%, or at least about 85%, or at least about 90% or of the period of time that the transducer is activated, and providing the second frequency for at most about 30%, or at most about 25%, or at most about 20%, or at most about 15%, or at most about 10% of the period of time that the transducer is activated. For example, the method may comprise providing the first frequency for about 90% of the period of time that the transducer is activated, and providing the second frequency for about 10% of the period of time that the transducer is activated.

According to one embodiment, the method comprises activating the transducer for about 500 ms, wherein during that time the signal provides the first frequency for about 90 ms and the second frequency for about 10 ms, e.g., the signal alternates five times between the first frequency for about 90 ms and the second frequency for about 10 ms. According to another embodiment, the method comprises activating the transducer for about 100 ms, wherein during that time the signal alternates between providing the first frequency for about 90 ms and providing the second frequency for about 10 ms.

According to one embodiment, the first frequency is from about 37 kHz to about 42 kHz, and the second frequency is either 36 kHz or less, or 43 kHz or more. For example, the second frequency may be from 0 kHz to about 30 kHz, or from about 45 kHz to about 75 kHz, or from about 50 kHz to about 60 kHz.

According to an embodiment, a method of treating a respiratory disease or disorder (e.g., COPD, asthma, cystic fibrosis, idiopathic pulmonary fibrosis, etc.) comprises using an embodiment of the inhaler described herein (e.g., by making consecutive inhalations through the inhaler) to administer a therapeutically effective amount of one or more medicaments.

The inhaler of the present invention is suitable for the delivery of many classes of medicaments by inhalation, and may be used for the treatment of various diseases and disorders. According to preferred embodiments, the inhaler is used for the treatment of respiratory disorders (e.g., COPD, asthma, cystic fibrosis, idiopathic pulmonary fibrosis, etc.). The inhaler may also be used to treat non-respiratory disorders.

According to particular embodiments, the methods described herein include methods for treating a respiratory disease or disorder amenable to treatment by respiratory delivery of a dry powder composition as described herein. For example, the compositions, methods and systems described herein can be used to treat inflammatory or obstructive pulmonary diseases or conditions. In certain embodiments, the compositions, methods and systems described herein can be used to treat patients suffering from a disease or disorder selected from asthma, chronic obstructive pulmonary disease (COPD), exacerbation of airways hyper reactivity consequent to other drug therapy, allergic rhinitis, sinusitis, pulmonary vasoconstriction, inflammation, allergies, impeded respiration, respiratory distress syndrome, pulmonary hypertension, pulmonary vasoconstriction, and any other respiratory disease, condition, trait, genotype or phenotype that can respond to the administration of, for example, a LAMA, LABA, corticosteroid, or other active agent as described herein, whether alone or in combination with other therapies. In certain embodiments, the compositions, systems and methods described herein can be used to treat pulmonary inflammation and obstruction associated with cystic fibrosis. As used herein, the terms “COPD” and “chronic obstructive pulmonary disease” encompass chronic obstructive lung disease (COLD), chronic obstructive airway disease (COAD), chronic airflow limitation (CAL) and chronic obstructive respiratory disease (CORD) and include chronic bronchitis, bronchiectasis, and emphysema. As used herein, the term “asthma” refers to asthma of whatever type or genesis, including intrinsic (non-allergic) asthma and extrinsic (allergic) asthma, mild asthma, moderate asthma, severe asthma, bronchitic asthma, exercise-induced asthma, occupational asthma and asthma induced following bacterial infection. Asthma is also to be understood as embracing wheezy-infant syndrome.

According to a preferred embodiment, the inhaler delivers one or more medicaments for the treatment of COPD; in particular, for the long-term, maintenance bronchodilator treatment of airflow obstruction in patients with chronic obstructive pulmonary disease (COPD), including chronic bronchitis and/or emphysema.

A range of classes of medicaments have been developed to treat respiratory disorders and each class has differing targets and effects.

Bronchodilators are employed to dilate the bronchi and bronchioles, decreasing resistance in the airways, thereby increasing the airflow to the lungs. Bronchodilators may be short-acting or long-acting. Typically, short-acting bronchodilators provide a rapid relief from acute bronchoconstriction, whereas long-acting bronchodilators help control and prevent longer-term symptoms.

Different classes of bronchodilators target different receptors in the airways. Two commonly used classes are anticholinergics and β2-agonists.

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Non-limiting examples of long-acting muscarinic antagonists (LAMA's) include tiotropium and pharmaceutically acceptable salts thereof (e.g., tiotropium bromide), oxitropium and pharmaceutically acceptable salts thereof (e.g., oxitropium bromide), aclidinium and pharmaceutically acceptable salts thereof (e.g., aclidinium bromide), ipratropium and pharmaceutically acceptable salts thereof (e.g., ipratropium bromide) glycopyrronium and pharmaceutically acceptable salts thereof (e.g., glycopyrronium bromide, also referred to as glycopyrrolate), oxybutynin and pharmaceutically acceptable salts thereof (e.g., oxybutynin hydrochloride or oxybutynin hydrobromide), tolterodine and pharmaceutically acceptable salts thereof (e.g., tolterodine tartrate), trospium and pharmaceutically acceptable salts thereof (e.g., trospium chloride), solifenacin and pharmaceutically acceptable salts thereof (e.g., solifenacin succinate), fesoterodine and pharmaceutically acceptable salts thereof (e.g., fesoterodine fumarate), darifenacin and pharmaceutically acceptable salts thereof (e.g., darifenacin hydrobromide) and umeclidinium and pharmaceutically acceptable salts thereof (e.g., umeclidinium bromide).

β2-Adrenergic agonists (or “β2-agonists”) act upon the β2-adrenoceptors and induce smooth muscle relaxation, resulting in dilation of the bronchial passages. Non-limiting examples of long-acting β2-adrenergic agonists (LABA's) include formoterol and pharmaceutically acceptable salts thereof (e.g., formoterol fumarate), salmeterol and pharmaceutically acceptable salts thereof (e.g., salmeterol xinafoate), indacaterol and pharmaceutically acceptable salts thereof (e.g., indacaterol maleate), bambuterol and pharmaceutically acceptable salts thereof (e.g., bambuterol hydrochloride), clenbuterol and pharmaceutically acceptable salts thereof (e.g., clenbuterol hydrochloride), olodaterol and pharmaceutically acceptable salts thereof (e.g., olodaterol hydrochloride), carmoterol and pharmaceutically acceptable salts thereof (e.g., carmoterol hydrochloride), tulobuterol and pharmaceutically acceptable salts thereof (e.g., tulobuterol hydrochloride) and vilanterol and pharmaceutically acceptable salts thereof (e.g., vilanterol triphenylacetate). Non-limiting examples of short-acting β2-agonists (SABA's) include albuterol and pharmaceutically acceptable salts thereof (e.g., albuterol sulfate) and levalbuterol and pharmaceutically acceptable salts thereof (e.g., levalbuterol tartrate). According to one embodiment, the formulation comprises albuterol (sulfate).

Another class of medicaments employed in the treatment of respiratory disorders are inhaled corticosteroids (ICS's). ICS's are steroid hormones used in the long-term control of respiratory disorders. They function by reducing the airway inflammation. Non-limiting examples of inhaled corticosteroids include budesonide and pharmaceutically acceptable salts thereof, beclomethasone and pharmaceutically acceptable salts thereof (e.g., beclomethasone dipropionate), fluticasone and pharmaceutically acceptable salts thereof (e.g., fluticasone propionate), mometasone and pharmaceutically acceptable salts thereof (e.g., mometasone furoate), ciclesonide and pharmaceutically acceptable salts thereof, and dexamethasone and pharmaceutically acceptable salts thereof (e.g., dexamethasone sodium).

According to an embodiment, the medicament delivery device delivers one or more medicaments selected from the group comprising or consisting of tiotropium and pharmaceutically acceptable salts thereof (e.g., tiotropium bromide), oxitropium and pharmaceutically acceptable salts thereof (e.g., oxitropium bromide), aclidinium and pharmaceutically acceptable salts thereof (e.g., aclidinium bromide), ipratropium and pharmaceutically acceptable salts thereof (e.g., ipratropium bromide) glycopyrronium and pharmaceutically acceptable salts thereof (e.g., glycopyrronium bromide, also referred to as glycopyrrolate), oxybutynin and pharmaceutically acceptable salts thereof (e.g., oxybutynin hydrochloride or oxybutynin hydrobromide), tolterodine and pharmaceutically acceptable salts thereof (e.g., tolterodine tartrate), trospium and pharmaceutically acceptable salts thereof (e.g., trospium chloride), solifenacin and pharmaceutically acceptable salts thereof (e.g., solifenacin succinate), fesoterodine and pharmaceutically acceptable salts thereof (e.g., fesoterodine fumarate), darifenacin and pharmaceutically acceptable salts thereof (e.g., darifenacin hydrobromide), umeclidinium and pharmaceutically acceptable salts thereof (e.g., umeclidinium bromide), formoterol and pharmaceutically acceptable salts thereof (e.g., formoterol fumarate), salmeterol and pharmaceutically acceptable salts thereof (e.g., salmeterol xinafoate), indacaterol and pharmaceutically acceptable salts thereof (e.g., indacaterol maleate), bambuterol and pharmaceutically acceptable salts thereof (e.g., bambuterol hydrochloride), clenbuterol and pharmaceutically acceptable salts thereof (e.g., clenbuterol hydrochloride), olodaterol and pharmaceutically acceptable salts thereof (e.g., olodaterol hydrochloride), carmoterol and pharmaceutically acceptable salts thereof (e.g., carmoterol hydrochloride), tulobuterol and pharmaceutically acceptable salts thereof (e.g., tulobuterol hydrochloride), vilanterol and pharmaceutically acceptable salts thereof (e.g., vilanterol triphenylacetate), albuterol and pharmaceutically acceptable salts thereof (e.g., albuterol sulfate), levalbuterol and pharmaceutically acceptable salts thereof (e.g., levalbuterol tartrate), beclomethasone and pharmaceutically acceptable salts thereof (e.g., beclomethasone dipropionate), fluticasone and pharmaceutically acceptable salts thereof (e.g., fluticasone propionate), mometasone and pharmaceutically acceptable salts thereof (e.g., mometasone furoate), ciclesonide and pharmaceutically acceptable salts thereof and dexamethasone and pharmaceutically acceptable salts thereof (e.g., dexamethasone sodium)and a combination thereof.

According to an embodiment, the medicament delivery device delivers a formulation comprising DNase (an enzyme that catalyzes cleavage of DNA), preferably DNase I or a variant thereof, most preferably human DNase I or a variant thereof. The DNase may be produced by known methods of recombinant DNA technology. The DNase may be administered for the treatment of a respiratory disease or disorder, such as cystic fibrosis (CF) or pneumonia. One type of DNase suitable for treating a respiratory disease or disorder is known as dornase alfa. The medicament delivery device preferably administers an amount of DNase that is effective to reduce the viscoelasticity of pulmonary secretions (mucus) in diseases such as CF or pneumonia, thereby aiding in the clearing of respiratory airways. As used herein, the term “human DNase I” refers to a polypeptide having the amino acid sequence of native human DNase I (see, e.g., SEQ. ID NO. 1 of U.S. Pat. No. 6,348,343). A “variant” of native human DNase I is a polypeptide having an amino acid sequence different from that of native human DNase I, e.g., at least 80% sequence identity (homology), preferably at least 90% sequence identity, more preferably at least 95% sequence identity, and most preferably at least 98% sequence identity with native human DNase I. The human DNase I or variant thereof exhibits DNA hydrolytic activity.

According to an embodiment, the medicament delivery device delivers a formulation comprising one or more antibiotics. The antibiotic(s) may be administered for the treatment of a respiratory disease or disorder, such as cystic fibrosis. Non-limiting examples of the classes of antibiotics that may be delivered by the medicament delivery device include tetracycline (e.g., doxycycline, minocycline, oxytetracycline, tigecycline), fluoroquinolone (e.g., ciprofloxacin, gemifloxacin, levofloxacin, moxifloxacin, norfloxacin, ofloxacin, sitafloxacin), carbapenem (e.g., meropenem, imipenem), polymyxin (e.g., colistin, polymyxin B) and combinations thereof. For example, a drug formulation may comprise an antibiotic selected from the group comprising or consisting of doxycycline, minocycline, oxytetracycline, tigecycline, ciprofloxacin, gemifloxacin, levofloxacin, moxifloxacin, norfloxacin, ofloxacin, sitafloxacin, meropenem, imipenem, colistin, polymyxin B and a combination thereof. A drug formulation may further comprise one or more adjuvants (potentiators of antibiotic activity) in combination with one or more antibiotics. According to an embodiment, a drug formulation comprises two or more antibiotics in combination, from the same class or different classes of antibiotic. A drug formulation may comprise one or more prodrugs of any of the aforementioned medicaments.

According to one embodiment, the medicament delivery device delivers a formulation comprising colistimethate sodium (a form of colistin) for the treatment of cystic fibrosis, or a formulation comprising doxycycline monohydrate for the treatment of cystic fibrosis, or a formulation comprising both colistimethate sodium and doxycycline monohydrate. According to another embodiment, the medicament delivery device delivers a formulation comprising pirfenidone for the treatment of idiopathic pulmonary fibrosis (IPF) or a symptom thereof.

According to particular embodiments, the inhaler delivers a combination of at least two different medicaments (two, three, four, etc.) which belong to the same or different classes. According to one embodiment, the medicament delivery device delivers a “triple combination” of three different medicaments. The three medicaments may belong to three different medicament classes (e.g., LAMA, LABA, ICS); alternatively, two or three of the medicaments may belong to the same class.

According to a preferred embodiment, the inhaler delivers one or more medicaments selected from the group comprising or consisting of a long-acting muscarinic antagonist (LAMA), a long-acting β2-adrenergic agonist (LABA) and a combination thereof. Thus, the medicament delivery device may deliver a formulation comprising one or more LAMA's in combination with one or more LABA's. A particularly suitable combination comprises glycopyrronium bromide (i.e., glycopyrrolate) and formoterol fumarate. Another suitable combination comprises tiotropium bromide and formoterol fumarate. Such combinations may be used for the treatment of COPD; in particular, for the long-term, maintenance bronchodilator treatment of airflow obstruction in patients with chronic obstructive pulmonary disease (COPD), including chronic bronchitis and/or emphysema. According to one embodiment, a combination of glycopyrrolate and formoterol fumarate, or tiotropium bromide and formoterol fumarate, is administered twice daily via oral tidal inhalation. Preferably, the combination achieves clinically significant bronchodilation vs. placebo at peak through trough (e.g., >100 ml), and/or significantly better bronchodilation (FEV₁) at peak through trough than monotherapy LABA (e.g., formoterol fumarate) or LAMA (e.g., glycopyrrolate or tiotropium bromide), and/or an onset of bronchodilation compared to placebo at 5 minutes after the first dose.

According to additional embodiments, the inhaler delivers one or more medicaments selected from the group comprising or consisting of a long-acting muscarinic antagonist (LAMA), a long-acting β2-adrenergic agonist (LABA), an inhaled corticosteroid (ICS) and a combination thereof. Thus, the medicament delivery device may deliver a formulation comprising one or more LAMA's, one or more LABA's and one or more ICS's. That is, the device may deliver a double combination of a LAMA and a LABA, a LAMA and an ICS, or a LABA and an ICS; or a triple combination of a LAMA, a LABA and an ICS.

Generally, as discussed herein, powdered medicament particles suitable for delivery to the bronchial or alveolar region of the lung have an aerodynamic diameter of less than 10 μm, preferably less than 6 μm. Other sized particles may be used if delivery to other portions of the respiratory tract is desired, such as the nasal cavity, mouth or throat. The medicament may be delivered as pure drug, but may alternatively be delivered together with one or more carriers and/or one or more excipients which are suitable for inhalation.

According to preferred embodiments, a powder formulation (also referred to herein as a “drug composition,” “composition,” “drug formulation,” “pharmaceutical composition,” “medicament formulation” or “API formulation”) comprises the medicament in combination with one or more carriers and/or one or more excipients. For example, a dose of medicament may be delivered in the form of a formulation comprising at least one medicament, at least one carrier (e.g., lactose) and optionally at least one excipient. According to particular embodiments, the inhaler contains a formulation dose in powder form, wherein each formulation dose comprises at least one medicament (e.g., a single medicament, or a combination of two medicaments, such as a LAMA and LABA), at least one carrier (e.g., lactose) and optionally at least one excipient (e.g., magnesium stearate). According to one example, the dose may comprise, consist essentially of, or consist of at least one medicament (e.g., a single medicament, or a combination of two medicaments, such as a LAMA and LABA) and a carrier (e.g., lactose) without any excipients.

Pharmaceutically acceptable carriers and excipients for dry powder formulations are known in the art. Lactose is a preferred carrier and magnesium stearate is a preferred excipient. Particles of a drug formulation may comprise surfactants, wall forming materials, or other components considered desirable by those of ordinary skill in the art. Particles of powdered medicament and/or powdered formulation may be produced by conventional techniques, for example by micronisation, milling, sieving or spray drying. Additionally, medicament and/or formulation powders may be engineered with particular densities, size ranges, or characteristics.

The drug formulations of the present invention are preferably propellant-free (e.g., free of propellant commonly used in inhalers, such as hydrofluoroalkane (HFA) propellant).

Embodiments of the present invention may be further understood by reference to the Examples provided below.

### EXAMPLES

Unless indicated otherwise, the medicament delivery device used in the examples below (e.g., “single-dose cartridge inhaler”) is an embodiment of the handheld device described herein, having a base and removable cartridge and powered by a rechargeable battery. A single dose of dry powder medicament is contained inside the dosing chamber, which is disposed in the cartridge. The piezoelectric transducer has a spacer of dielectric ink screen printed on its face (e.g., Acheson ML25240 UV Cure Dielectric Ink, electrically non-conductive ink) in the pattern of a discontinuous ring positioned at or near the perimeter of the transducer face. The nominal spacer thickness applied to the face of the piezo is about 53 μm±25 μm. The piezo is pressed against the dosing chamber membrane via a mounting system comprising a holder and spring. The aluminum piezo is driven at a resonant frequency between 38-42 kHz with a hop frequency of about 54 kHz and voltage of 200-240 V p-p. The membrane is co-extruded polyethylene terephthalate (PET, DuPont Mylar® 813) with one side heat sealable amorphous PET, having a nominal thickness of about 23 μm±10 μm. The dosing chamber has four openings in the apex with diameters of 0.019 inches (0.48 mm) 0.012 inches (0.30 mm). The flow resistance is between 0.050-0.1 cmH₂O^{0.5}/LPM at a flow rate of 30 LPM. For in vitro tests described below, unless indicated otherwise, a flow rate of 30 LPM was used.

All aerodynamic particle size distributions (APSD) were determined using a Next Generation Impactor (NGI). Samples were analyzed using single-point calibration on an HPLC system with UV detection at 220 nm.

### Example 1: Synthetic Jetting Test Procedure

Reference: Service and Instruction Manual, Rudolph Pneumotachometers (PNT) and Heater Controllers ISO 9001/ISO 13485.

Materials and Equipment:
Linear Pneumotachometer 3500 Series 0-35 L/min by Hans Rudolph, Inc. (or equivalent)
Pneumotach Amplifier 1 Series 1110 by Hans Rudolph, Inc. (or equivalent)
Digital Storage Oscilloscope (or equivalent)
Inhaler Subassembly with Aerosol Engine comprising jetting fixture (or equivalent)
Breakout Board and Flat Flex Jumper Assembly S0363 (or equivalent)
Remote Start Switch (or equivalent)
BNC Coaxial Cable (or equivalent)
Ribbon Insertion Tool S0627 (or equivalent)
Connector Latch Tool P2767 (or equivalent)

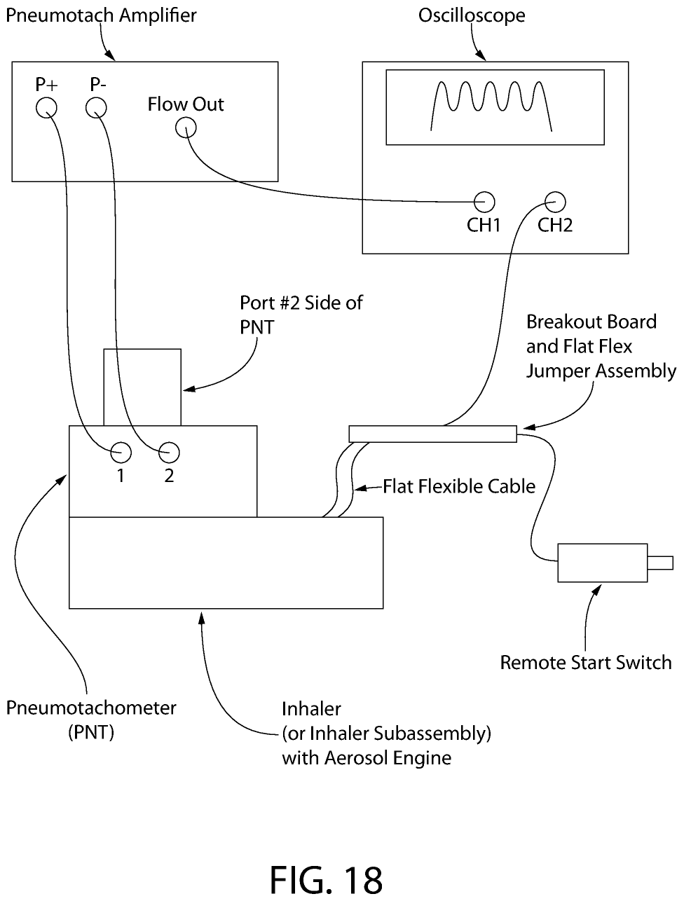

An example of the Equipment Setup is illustrated in FIG. 18. A flat flex cable (FFC) provides control and feedback signals so that the jetting signal can be aligned with the piezoelectric transducer firing on the oscilloscope. The pneumotachometer is preferably installed so that the net jetting flow out of the mouthpiece port generates a positive signal on the oscilloscope. The PNT is positioned over the dosing chamber holes using the mask port, and captures the net flow exiting all the hole(s) of the dosing chamber. The net flow is the cumulative effect of the outward momentum of each of the individual jets occurring at the piezo drive frequency (e.g., approximately 37-42 kHz).

Equipment Setup Example:
1. Connect the Flat Flexible Cable (FFC) jumper locking lever to the inhaler. A ribbon insertion tool may be used to guide the FFC into the inhaler. The blue insulator on the end of the cable should be facing the inhaler. A locking lever may be used to lock the FFC in place. The aerosol engine/jetting fixture should be clamped securely in place.
2. Attach the pneumotachometer (PNT) to the inhaler. The Port #2 side of the PNT should be facing away from the device.
3. Attach the PNT tubing to connect the pneumotachometer to the pneumotach amplifier. The tubing with a white label should be attached to the PNT input labeled “1” and the “P+” input on the pneumotach amplifier. The tubing with a black label should be attached to the PNT input labeled “2” and the “P−” input on the amplifier.
4. Use a BNC coaxial cable to connect the amplifier “Flow Out” to “CH1” on the oscilloscope.
5. Run the oscilloscope in the following setting:
   a. Time Mode: Roll
6. Verify the baseline voltage is at zero. If not, use a screwdriver to adjust the “ZERO” setting on the pneumotach amplifier until a zero voltage reading is displayed.
7. Connect the coaxial cable attached to the breakout board TP1 and GND pins to “CH2” on the oscilloscope.
8. Adjust the oscilloscope settings as follows:
   a. CH1: 50 mV/div with a 150 mV offset
   b. CH2: 200 mV/div with a 600 mV offset
   c. Time Mode: Normal with 100 ms hold off
   d. TRIG: CH2 rising edge at a 850 mV level
   e. HORZ: 5 ms/div with a 10 ms left position delay
9. Press the “Quick Measure” button on the oscilloscope and select to measure the Source 1 Pk-Pk voltage.
10. Connect the remote start switch to the breakout board SW1 and GND pins.
11. Press the remote start switch to turn on the device. Verify the device has powered on by observing the light-up sequence display on the device overlay.
12. Press and hold the remote start switch for at least 5 seconds until the device triggers. When this happens a trace will appear on the oscilloscope.
13. Record the Pk-Pk (1) voltage as the peak PNT signal.
14. Repeat steps 11-13 as required.

### Example 2: Test procedure for Determining Flow Resistance of Air Flow Conduit

References, each of which is incorporated by reference herein in their entirety:
1. United States Pharmacopia General Chapters <601> Aerosols, Nasal Sprays, Meter-Dose Inhalers, and Dry Powder Inhalers;
2. “Testing Inhalers” David Harris, Pharmaceutical Technology Europe, September 2007, pg 29-35;
3. A. R. Clarke and A. M. Hollingworth, J. Aerosol Med., 6 99-110 (1993).

Materials and Equipment:
1. Inhaler air flow conduit and mouthpiece adapter to the testing apparatus volume (or equivalent);
2. Air flow conduit adapter chamber with pressure port P1 part #1987A as part of Subassembly S0417A (or equivalent);
3. Differential Pressure Meter—Digitron Model #2020P or 2000P for 0-10″ W.C. range and Model #2022P for >10″ W.C. (or equivalent)
4. Flow Meter—Cole Parmer Model #32908-75 (or equivalent)
5. Flow Control Valve with a Cv≥1.0—Parker Hannifin type 8F-V12LN-SS (or equivalent)
6. Vacuum Pump—Gast Type 1023, 1423 or 2565 (or equivalent)
7. Tubing—Tygon B-44-4X 10 mm ID and Tygon 4 mm (5/32′) ID (or equivalent)

Procedure:

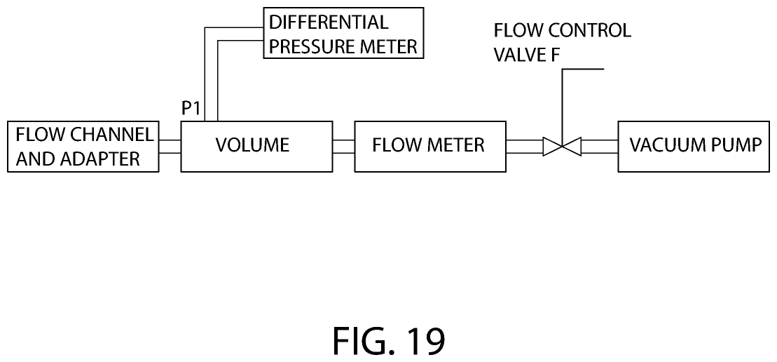

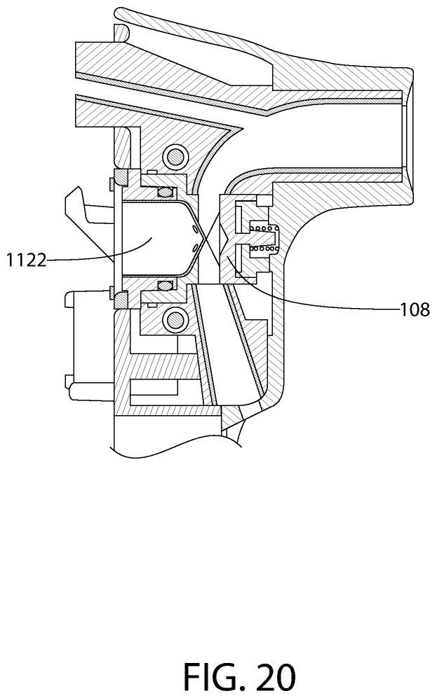

Set up the system with the diagram shown in FIG. 19.
1. Apply power to both the Flow Meter and Pressure Sensor and allow 10 minutes for warm-up. After warm-up, zero both the pressure sensor and flow meter.
2. Ensure tight air seals on all connections. When the thumb is placed over the opening at the air flow conduit adapter chamber, the Flow Meter should read zero.
3. To measure the inhaler flow resistance, insert the inhaler into the air flow conduit adapter chamber. Turn on the vacuum pump and adjust Flow Control valve F until the flow meter reads the required tested flow rate. Record the pressure differential (PI) from the Differential Pressure Meter in inches W.C.; convert to cm W.C.
4. Calculate the inhaler flow resistance using the following equation:

$$\begin{matrix}Flow Resistance = Square Root ( Pressure in cm W . C . ) \text{/} \\ Flow Rate in L \text{/} min \\ = Square Root {( P 1 \times 2.54 )}^{*} \text{/} flow rate \\ = cm {H}_{2} {O}^{1 / 2} \cdot {( L \text{/} min )}^{- 1}\end{matrix}$$

*Conversion from inches to centimeters; 1 inch=2.54 cm

### Example 3: Aerosol Performance of Large Biologic Molecule in Single-Dose Cartridge

Aerosol performance of a spray-dried enzyme (with excipients) was tested in the inhaler with a single-dose cartridge at two concentrations of the enzyme (17.5 and 50% w/w) and two fill weights in the dosing chamber (5 mg and 12 mg). The enzyme was a DNase known as dornase alfa (recombinant human deoxyribonuclease I). Each dose was contained in the dosing chamber of a single-dose cartridge. The device was programmed to activate the piezo for 8 timed bursts of 500 ms duration and the flow rate was 30 LPM. Results are shown in Table 1 below. The inhaler demonstrated consistent performance across varying API concentrations and fill weights. The fine particle fraction (FPF) was greater than 50%, e.g., between about 60% and about 70%, and MMAD was less than 5.0 microns, e.g., between about 3.0 microns to about 4.0 microns for all three formulations.

TABLE 1

| | Spray-Dried Biologic | | |
|---|---|---|---|
| Formulation | Compound (50% w/w) | Spray-Dried Biologic Compound (17.5% w/w) | |
| Fill Weight (mg) | 5 | 5 | 12 |
| Loaded Dose (mg) | 2.33 | 0.89 | 2.10 |
| | (range: 2.16-2.48) | (range: 0.82-2.48) | (range: 2.04-2.48) |
| | | Compound (17.5% | |
| % Dose Chamber | 92.4 | 96.9 | 95.2 |
| Clearance | (range: 83.7-109.0) | (range: 92.7-98.4) | (range: 92.8-101.8) |
| Fine Particle Dose (mg) | 1.03 | 0.54 | 1.07 |
| | (range: 0.52-1.21) | (range: 0.48-0.61) | (range: 1.04-1.09) |
| Fine Particle Fraction | 68.0 | 64.7 | 62.7 |
| | (range: 65.1-72.6) | (range: 60.2-67.6) | (range: 60.8-66.5) |
| MMAD (microns) | 3.3 | 3.5 | 3.8 |
| | (range: 3.2-3.4) | (range: 3.4-3.8) | (range: 3.6-3.9) |

### Example 4: Aerosol Performance of Small Molecule in Single-Dose Cartridge

Aerosol performance of a small molecule compound spray-dried with an excipient (about 80% w/w) was tested in the inhaler with a single-dose cartridge at five different fill weights (8, 16, 28, 40 and 50 mg). Each dose was contained in the dosing chamber of a single-dose cartridge. The device was programmed to activate the piezo for 8 timed bursts of 500 ms duration and the flow rate was 30 LPM. Results are shown in Table 2 below. The inhaler demonstrated consistent performance with fill weights from 8 mg through 50 mg. The fine particle fraction (FPF) was greater than 50%, e.g., between about 60% and about 70%, and MMAD was less than 5.0 microns, e.g., between about 3.0 microns to about 4.0 microns for all three formulations.

TABLE 2

| | | | | | |
|---|---|---|---|---|---|
| Fill Weight (mg) | 8 | 16 | 28 | 40 | 50 |
| Metered Dose (mg) | 6.5 | 12.9 | 22.7 | 32.4 | 40.4 |
| Delivered Dose (mg) | 4.24 | 8.44 | 13.96 | 19.94 | 24.88 |
| Fine Particle Dose (mg) | 2.87 | 5.54 | 9.34 | 13.86 | 16.63 |
| % Fine Particle Fraction | 68 | 66 | 67 | 69 | 67 |
| MMAD | 3.3 | 3.5 | 3.4 | 3.3 | 3.4 |

It will be appreciated by those skilled in the art that changes may be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Selected features of any illustrative embodiment may be incorporated into an additional embodiment unless clearly stated to the contrary. The words “right”, “left”, “lower” and “upper” designate directions in the drawings to which reference is made. The words “inwardly” and “outwardly” refer to directions toward and away from, respectively, the geometric center of the inhaler. Unless specifically set forth herein, the terms “a”, “an” and “the” are not limited to one element but instead should be read as meaning “at least one”. Elements shown in the Figures are not necessarily drawn to scale, but only to illustrate operation.

As used herein and in the claims, the terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.” Unless specified otherwise, all values provided herein include up to and including the endpoints given, and the values of the constituents or components of the compositions are expressed in weight percent of each ingredient in the composition.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

I/We claim:

1. A medicament delivery device comprising:
a dosing chamber comprising an interior that is configured to contain dry powder medicament,
a transducer confronting the dosing chamber, wherein the dosing chamber and the transducer are acoustically resonant such that the dosing chamber is configured to resonate in response to an activation of the transducer, and
a controller electrically coupled to the transducer and configured to send an electrical signal that activates the transducer when the medicament delivery device senses a subject's dosing breath,
the medicament delivery device having a flow resistance in a range from about 0.040 cmH₂O^{0.5}/LPM to about 0.1 cmH₂O^{0.5}/LPM at 30 liters per minute (LPM) and capable of delivering a therapeutically effective dose of dry powder medicament in response to between 2-20 tidal inhalations,
wherein the medicament delivery device comprises a base and a removable single-dose cartridge, the dosing chamber is contained within the removable single-dose cartridge, and the dose of dry powder medicament is contained inside the dosing chamber.

2. The medicament delivery device of claim 1, wherein the controller is configured to activate the transducer for a total on-time of 5 seconds or less over the 2-20 tidal inhalations.

3. The medicament delivery device of claim 1, wherein the controller is configured to activate the transducer for between 50 ms and about 1000 ms during each dosing breath.

4. The medicament delivery device of claim 1, wherein the medicament delivery device is capable of delivering the dose of dry powder medicament at flow rates at least within a range of about 15 LPM to about 30 LPM.

5. The medicament delivery device of claim 1, wherein the medicament delivery device is capable of delivering the dose of dry powder medicament at flow rates at least within a range of about 15 LPM to about 90 LPM.

6. The medicament delivery device of claim 1, wherein the medicament delivery device is configured to administer at least 10% of the dry powder medicament dose in response to a first dosing breath.

7. The medicament delivery device of claim 1, wherein the dose has a mass median aerodynamic diameter (MMAD) of about 6 microns or less and a fine particle fraction of at least 30%.

8. The medicament delivery device of claim 1, wherein the amount of the dose of dry powder medicament is between about 1 mg and about 100 mg.

9. The medicament delivery device of claim 1 comprising one or more lights configured to illuminate when a dose has been administered.

10. A method of treating a respiratory disease or condition, or one or more symptoms thereof, the method comprising:
inhaling a therapeutically effective dose of dry powder medicament through a medicament delivery device using between 2-20 tidal inhalations over the course of an inhalation cycle, the inhalation cycle comprising dosing breaths,
wherein the medicament delivery device comprises a vibratory element that is activated upon each dosing breath and causes dry powder medicament to be aerosolized within a dosing chamber and expelled from one or more openings in the dosing chamber into an air flow conduit, wherein pressure oscillations in the dosing chamber are sufficiently high at the one or more openings to aerosolize and expel the dry powder medicament via synthetic jetting,
wherein the medicament delivery device comprises a base and a removable single-dose cartridge, the dosing chamber is contained within the removable single-dose cartridge, and the dose of dry powder medicament is contained inside the dosing chamber,
the medicament delivery device having a flow resistance in a range from about 0.040 cmH₂O^{0.5}/LPM to about 0.1 cmH₂O^{0.5}/LPM at 30 liters per minute (LPM) and capable of delivering the dose of dry powder medicament at flow rates at least within a range of about 15 LPM to about 30 LPM.

11. The method of claim 10 further comprising exhaling away from the medicament delivery device after each tidal inhalation.

12. The method of claim 10, wherein the dose of dry powder medicament administered by the medicament delivery device has a mass median aerodynamic diameter (MMAD) of about 6 microns or less and a fine particle fraction of at least 30%.

13. The method of claim 10, wherein the medicament delivery device administers at least 10% of the dry powder medicament dose in response to the first dosing breath in the inhalation cycle.

14. The method of claim 10, wherein the transducer has an on-time of about 5 seconds or less over the course of the inhalation cycle.

15. The method of claim 10, wherein the medicament delivery device achieves maximum synthetic jetting within about 1000 ms or less from the start of each transducer activation.

16. The method of claim 10, wherein the dose of dry powder medicament is administered within 2 minutes or less.

17. The method of claim 10, wherein the respiratory disease or condition is COPD.

18. The method of claim 10, wherein the respiratory disease or condition is COPD and the dry powder medicament comprises a LAMA and a LABA.

19. The method of claim 10, wherein the respiratory disease or condition is COPD and the dry powder medicament comprises glycopyrronium bromide and formoterol fumarate.

20. The method of claim 10, wherein the respiratory disease or condition is asthma.

21. The method of claim 10, wherein the respiratory disease or condition is cystic fibrosis and the dry powder medicament comprises one or more antibiotics.

22. The method of claim 10, wherein the respiratory disease or condition is cystic fibrosis and the dry powder medicament comprises DNase.

23. The method of claim 10, wherein the respiratory disease or condition is idiopathic pulmonary fibrosis and the dry powder medicament comprises pirfenidone.

24. A method of increasing FEV₁ in a subject, the method comprising:
inhaling a therapeutically effective dose of dry powder medicament through a medicament delivery device using between 2-20 tidal inhalations over the course of an inhalation cycle, the inhalation cycle comprising dosing breaths,
wherein the medicament delivery device comprises a vibratory element that is activated upon each dosing breath and causes dry powder medicament to be aerosolized within a dosing chamber and expelled from one or more openings in the dosing chamber into an air flow conduit, wherein pressure oscillations in the dosing chamber are sufficiently high at the one or more openings to aerosolize and expel the dry powder medicament via synthetic jetting,
wherein the medicament delivery device comprises a base and a removable single-dose cartridge, the dosing chamber is contained within the removable single-dose cartridge, and the dose of dry powder medicament is contained inside the dosing chamber,
the medicament delivery device having a flow resistance in a range from about 0.040 cmH₂O^{0.5}/LPM to about 0.1 cmH₂O^{0.5}/LPM at 30 liters per minute (LPM) and capable of delivering the dose of dry powder medicament at flow rates at least within a range of about 15 LPM to about 30 LPM.

25. The method of claim 24, wherein the medicament delivery device administers at least 10% of the dry powder medicament dose in response to the first dosing breath in the inhalation cycle.

26. The method of claim 24, wherein the transducer has an on-time of about 5 seconds or less over the course of the inhalation cycle.

27. The method of claim 24, wherein the medicament delivery device achieves maximum synthetic jetting within about 1000 ms or less from the start of each transducer activation.

28. The method of claim 24, wherein the dose of dry powder medicament delivered by the medicament delivery device has a mass median aerodynamic diameter (MMAD) of about 6 microns or less and a fine particle fraction of at least 30%.

29. The method of claim 24, wherein the amount of the dose of dry powder medicament is between about 1 mg and about 100 mg.

30. A method of treating a respiratory disease or condition, or one or more symptoms thereof, the method comprising:
inhaling a therapeutically effective dose of dry powder medicament through a medicament delivery device using between 2-20 tidal inhalations over the course of an inhalation cycle, the inhalation cycle comprising dosing breaths,
wherein the medicament delivery device comprises a vibratory element that is activated upon each dosing breath and causes dry powder medicament to be aerosolized within a dosing chamber and expelled from one or more openings in the dosing chamber into an air flow conduit, wherein pressure oscillations in the dosing chamber are sufficiently high at the one or more openings to aerosolize and expel the dry powder medicament via synthetic jetting,
wherein the medicament delivery device comprises a base and a removable single-dose cartridge, the dosing chamber is contained within the removable single-dose cartridge, and the dose of dry powder medicament is contained inside the dosing chamber,
wherein the air flow conduit comprises a constricted section disposed over the one or more openings in the dosing chamber, or adjacent to the one or more openings in the dosing chamber,
wherein the dose of medicament is expelled over the course of the inhalation cycle by a combination of active delivery and passive delivery, and
wherein the medicament delivery device is capable of delivering the dose of dry powder medicament at flow rates at least within a range of about 15 LPM to about 30 LPM.
